(19) United States
(12) Patent Application Publication
(10) Pub. No.: US 2008/0015659 A1
(43) Pub. Date: Jan. 17, 2008

(54) NEUROSTIMULATION SYSTEMS AND METHODS FOR CARDIAC CONDITIONS

(76) Inventors: Yi Zhang, Blaine, MN (US); Anthony V. Caparso, San Jose, CA (US); Imad Libbus, St. Paul, MN (US); Aaron McCabe, Minneapolis, MN (US); Scott A. Meyer, Lakeville, MN (US); Marina V. Brockway, Shoreview, MN (US)

Correspondence Address: SCHWEGMAN, LUNDBERG & WOESSNER, P.A., P.O. BOX 2938, MINNEAPOLIS, MN, 55402, US

(21) Appl. No.: 11/860,936

(22) Filed: Sep. 25, 2007

Related U.S. Application Data
(63) Continuation-in-part of application No. 10/745,925, filed on Dec. 24, 2003.
(63) Continuation-in-part of application No. 11/207,251, filed on Aug. 19, 2005.

(51) Int. Cl. A61N 1/36

(52) U.S. Cl. 607/62

(57) ABSTRACT

Various embodiments provide an implantable medical device comprising a detector, a neural stimulator, and a controller. The detector is configured to detect a pathological condition indicated for an acute neural stimulation therapy. The neural stimulator is capable of delivering a chronic neural stimulation therapy and the acute neural stimulation therapy. The controller is configured to control the neural stimulator to provide the chronic neural stimulation therapy, receive an indicator from the detector that the pathological condition is detected, and control the neural stimulator to integrate the acute neural stimulation therapy with the chronic neural stimulation therapy in response to the indicator.

### CROSS REFERENCE TO RELATED APPLICATIONS

This application is a continuation-in-part application of U.S. patent application Ser. No. 10/745,925, filed on Dec. 24, 2003, entitled “Automatic Baroreflex Modulation Responsive to Adverse Event,” published as US 2005/0149127, and a continuation-in-part application of U.S. patent application Ser. No. 11/207,251, filed on Aug. 19, 2005, entitled “Method and Apparatus For Delivering Chronic and Post-Ischemia Cardiac Therapies,” published as US 2007/0043393, which are herein incorporated by reference in their entirety.

### TECHNICAL FIELD

This application relates generally to medical devices and, more particularly, to systems, devices and methods for providing neural stimulation therapies for cardiac conditions.

### BACKGROUND

Implanting a chronic electrical stimulator, such as a cardiac stimulator, to deliver medical therapy(ies) is known. Examples of cardiac stimulators include implantable cardiac rhythm management (CRM) devices such as pacemakers, implantable cardiac defibrillators (ICDs), and implantable devices capable of performing pacing and defibrillating functions.

CRM devices are implantable devices that provide electrical stimulation to selected chambers of the heart in order to treat disorders of cardiac rhythm. An implantable pacemaker, for example, is a CRM device that paces the heart with timed pacing pulses. If functioning properly, the pacemaker makes up for the heart's inability to pace itself at an appropriate rhythm in order to meet metabolic demand by enforcing a minimum heart rate. Some CRM devices synchronize pacing pulses delivered to different areas of the heart in order to coordinate the contractions. Coordinated contractions allow the heart to pump efficiently while providing sufficient cardiac output.

It has been proposed to stimulate neural targets to treat a variety of pathological conditions. For example, research has indicated that electrical stimulation of the carotid sinus nerve can result in reduction of experimental hypertension, and that direct electrical stimulation to the pressoreceptive regions of the carotid sinus itself brings about reflex reduction in experimental hypertension.

Myocardial infarction (MI) is the necrosis of portions of the myocardial tissue resulted from cardiac ischemia, a condition in which the myocardium is deprived of adequate oxygen and metabolite removal due to an interruption in blood supply caused by an occlusion of a blood vessel such as a coronary artery. The necrotic tissue, known as infarcted tissue, loses the contractile properties of the normal, healthy myocardial tissue. Consequently, the overall contractility of the myocardium is weakened, resulting in an impaired hemodynamic performance. Following an MI, cardiac remodeling starts with expansion of the region of infarcted tissue and progresses to a chronic, global expansion in the size and change in the shape of the entire left ventricle. The consequences include a further impaired hemodynamic performance, higher risk of ventricular arrhythmia, and a significantly increased risk of developing heart failure.

### SUMMARY

Various embodiments improve cardiac neural therapy after a detected pathological cardiac event. Various embodiments improve cardiac function and control remodeling following ischemic events or an acute MI. For a patient who has been receiving a neural stimulation therapy on a long-term basis (referred to herein as chronic neural stimulation therapy) prior to the occurrence of such a pathological cardiac event, there is a need to adjust the therapeutic strategy in response to the pathological cardiac event.

Various embodiments provide an implantable medical device comprising a detector, a neural stimulator, and a controller. The detector is configured to detect a pathological condition indicated for an acute neural stimulation therapy. The neural stimulator is capable of delivering a chronic neural stimulation therapy and the acute neural stimulation therapy. The controller is configured to control the neural stimulator to provide the chronic neural stimulation therapy, receive an indicator from the detector that the pathological condition is detected, and control the neural stimulator to integrate the acute neural stimulation therapy with the chronic neural stimulation therapy in response to the indicator.

In a method embodiment, a first neural stimulation therapy is performed to treat a first pathological condition. A second pathological condition is detected. The second pathological condition is a cardiac condition indicated for a second neural stimulation therapy. In response to detecting the second pathological condition, the first neural stimulation therapy and the second neural stimulation therapy are integrated into an integrated neural stimulation therapy for the first and second pathological conditions.

In a method embodiment, a vagal stimulation therapy is performed to treat a chronic pathological condition. When ischemia is detected, the vagal stimulation therapy is adjusted for the chronic pathological condition in response to detecting the ischemia.

This Summary is an overview of some of the teachings of the present application and not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF THE DRAWINGS

FIGS. 1A and 1B illustrate neural mechanisms for peripheral vascular control.

FIGS. 2A-2C illustrate a heart.

FIG. 3 illustrates baroreceptors in the area of the carotid sinuses, aortic arch and pulmonary artery.

FIG. 4 illustrates baroreceptors in and around a pulmonary artery.

FIG. 5 illustrates baroreceptor fields in the aortic arch.

FIG. 6 illustrates a known relationship between respiration and blood pressure when the left aortic nerve is stimulated.

FIG. 7 illustrates a known blood pressure response to carotid nerve stimulation in a hypertensive dog during 6 months of intermittent carotid nerve stimulation.

FIG. 8 illustrates a system embodiment including an implantable medical device (IMD) and a programmer.

FIG. 9 illustrates a system embodiment including a programmer, an implantable neural stimulator (NS) device and an implantable cardiac rhythm management (CRM) device.

FIG. 10 illustrates an embodiment of an implantable medical device (IMD).

FIG. 11 illustrates an embodiment of an implantable medical device (IMD) having a neural stimulation (NS) component and a cardiac rhythm management (CRM) component.

FIG. 12 shows a system diagram of an embodiment of a microprocessor-based implantable device.

FIG. 13 is a block diagram illustrating an embodiment of an external system.

FIG. 14 illustrates a system embodiment in which an implantable medical device (IMD) is placed subcutaneously or submuscularly in a patient's chest with lead(s) positioned to stimulate a neural target in the cervical region.

FIG. 15 illustrates a system embodiment that includes an implantable medical device (IMD) with satellite electrode(s) positioned to stimulate at least one cervical neural target.

FIG. 16 illustrates an IMD placed subcutaneously or submuscularly in a patient's chest with lead(s) positioned to provide a CRM therapy to a heart, and with lead(s) positioned to stimulate and/or inhibit neural traffic at a cervical neural target, according to various embodiments.

FIG. 17 illustrates an IMD with lead(s) positioned to provide a CRM therapy to a heart, and with satellite transducers positioned to stimulate/inhibit a cervical neural target, according to various embodiments.

FIG. 18 illustrates a device embodiment configured to integrate neural stimulation therapies for at least two detected pathological conditions.

FIG. 19 illustrates a device embodiment configured to integrate a chronic neural stimulation therapy for a chronic pathological condition with a neural stimulation therapy for a detected, acute pathological condition.

FIGS. 20-21 illustrate methods for modulating baroreceptor stimulation based on detection of a detected cardiac event, according to various embodiments of the present subject matter.

FIGS. 22-23 illustrate a system and method to detect myocardial infarction and perform baropacing in response to the detected myocardial infarction, according to various embodiments of the present subject matter.

FIG. 24 is a block diagram illustrating an embodiment of a pre-ischemia and post-ischemia therapy system.

FIG. 25 is an illustration of an embodiment of an electrode system for detecting the ischemic state and/or locating the ischemic region using electrograms and/or impedance signals.

FIG. 26 is an illustration of an embodiment of an electrode/sensor system for detecting the ischemic event and/or locating the ischemic region.

FIG. 27 illustrates a method embodiment for delivering a chronic neural stimulation therapy and a post-ischemia neural stimulation therapy.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments treat myocardial ischemia and/or infarction using neurostimulation therapy. The occurrence of a myocardial ischemia/infarction event is detected, and electrical stimulation to one or more predetermined neural targets in the patient is delivered. Various embodiments use a real-time ischemia detection algorithm to initiate, titrate, modify neurostimulation therapy and monitor its effectiveness.

An acute MI event can be detected through many different ways (e.g. wireless ECG, EGM, heart sounds, pressure, impedance, heart rate variability (HRV), etc.) In various embodiments, neural stimulation therapy is adjusted in response to the detection of an infarct, or an abnormal surge in sympathetic activity. Intracardiac impedance can be used to differentiate between ischemic and infarcted tissue. Neural stimulation therapy can be delivered using various neural targets, such as baroreceptors, carotid sinus, cardiac branch of the vagal trunk or the vagus nerve, etc. Neural stimulation therapy can be delivered to control heart rate, contractility, conduction velocity, or atrioventricular (AV) delay to release the stress caused by an acute MI event. The effectiveness of the neural stimulation therapy can be monitored through heart rate, heart sounds, HRV, etc. Neural stimulation therapy can be titrated by controlling the frequency of the stimulation, amplitude, duty cycle, waveform, stimulation site, etc.

The present subject matter can be used for any patient at high risk for ischemic events. The device could be a standalone implantable neural stimulator, or a combined device to provide neural stimulation therapy, and cardiac rhythm management. If ischemic detection is accomplished with an intravascular cardiac lead, neural stimulation can be also delivered transvascularly, such as with a lead positioned to transvascularly stimulation the vagus nerve or a cardiac fat pad. If ischemic detection is accomplished through subcutaneous means, neural stimulation can be delivered directly to the appropriate neural target, such as with a cuff electrode placed around the nerve trunk, or a subcutaneous lead placed in the vicinity of the neural target. The device can be an external unit that detects an ischemic event and provides transcutaneous stimulation to an appropriate neural target.

Provided below, for the benefit of the reader, is a brief discussion of physiology and therapies. The disclosure continues with a discussion of various system embodiments and corresponding devices and methods.

### Physiology

The autonomic nervous system (ANS) regulates “involuntary” organs, while the contraction of voluntary (skeletal) muscles is controlled by somatic motor nerves. Examples of involuntary organs include respiratory and digestive organs, and also include blood vessels and the heart. Often, the ANS functions in an involuntary, reflexive manner to regulate glands, to regulate muscles in the skin, eye, stomach, intestines and bladder, and to regulate cardiac muscle and the muscle around blood vessels, for example.

The ANS includes the sympathetic nervous system and the parasympathetic nervous system. The sympathetic nervous system is affiliated with stress and the “fight or flight response” to emergencies. Among other effects, the “fight or flight response” increases blood pressure and heart rate to increase skeletal muscle blood flow, and decreases digestion to provide the energy for “fighting or fleeing.” The parasympathetic nervous system is affiliated with relaxation and the “rest and digest response” which, among other effects, decreases blood pressure and heart rate, and increases digestion to conserve energy. The ANS maintains normal internal function and works with the somatic nervous system.

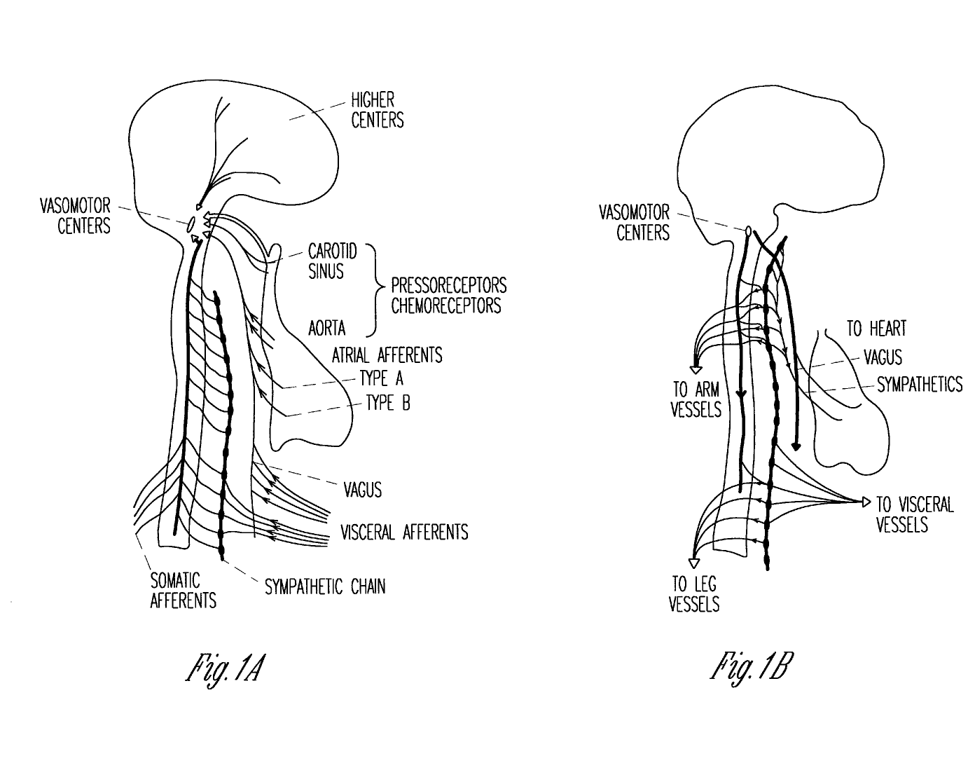

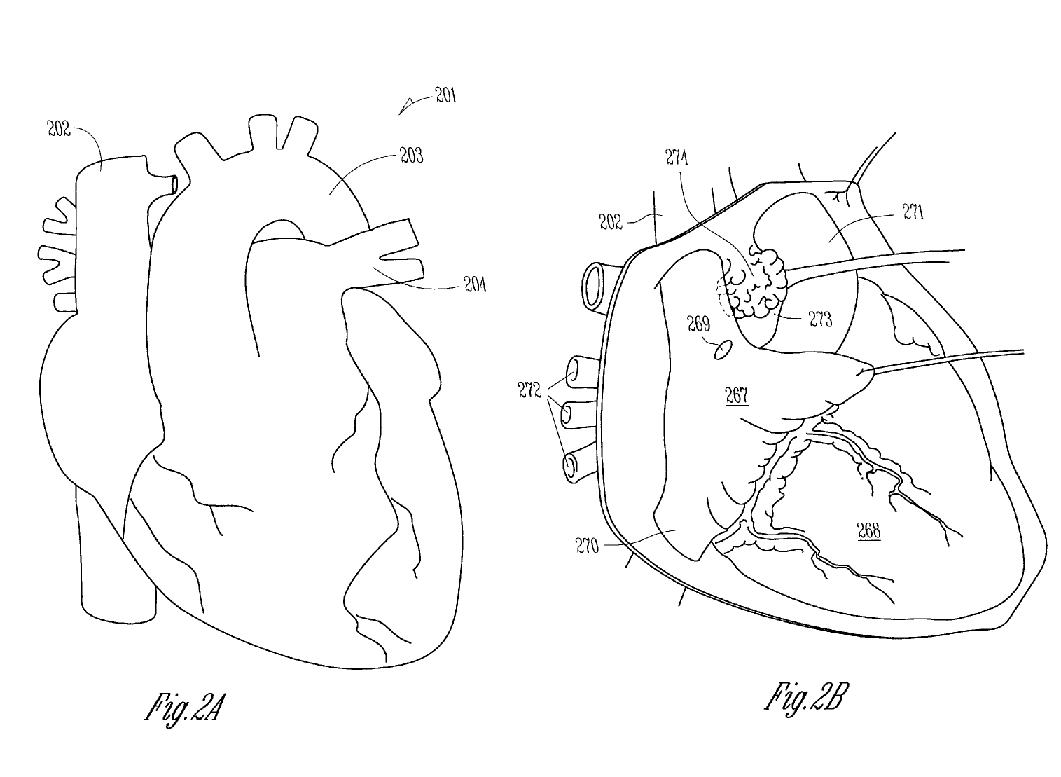

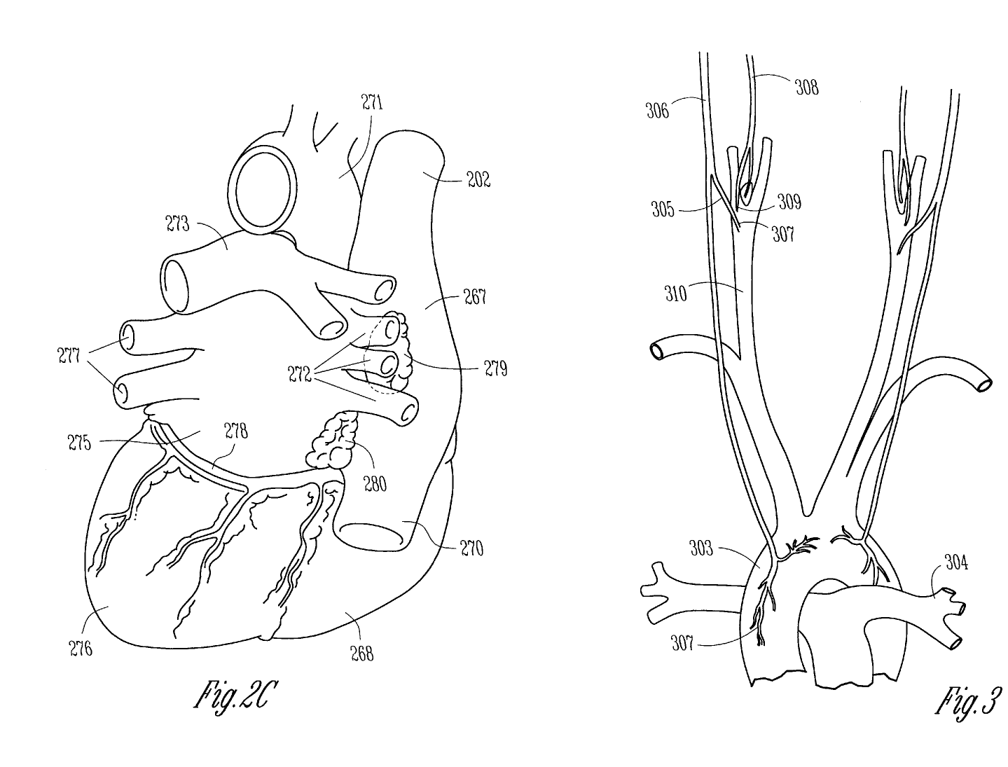

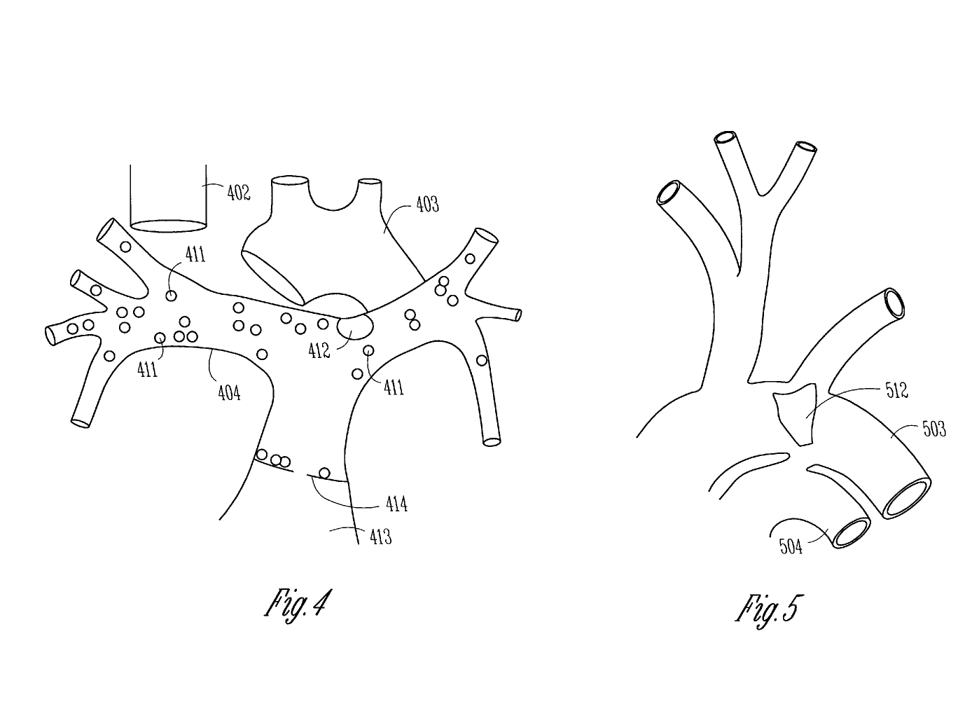

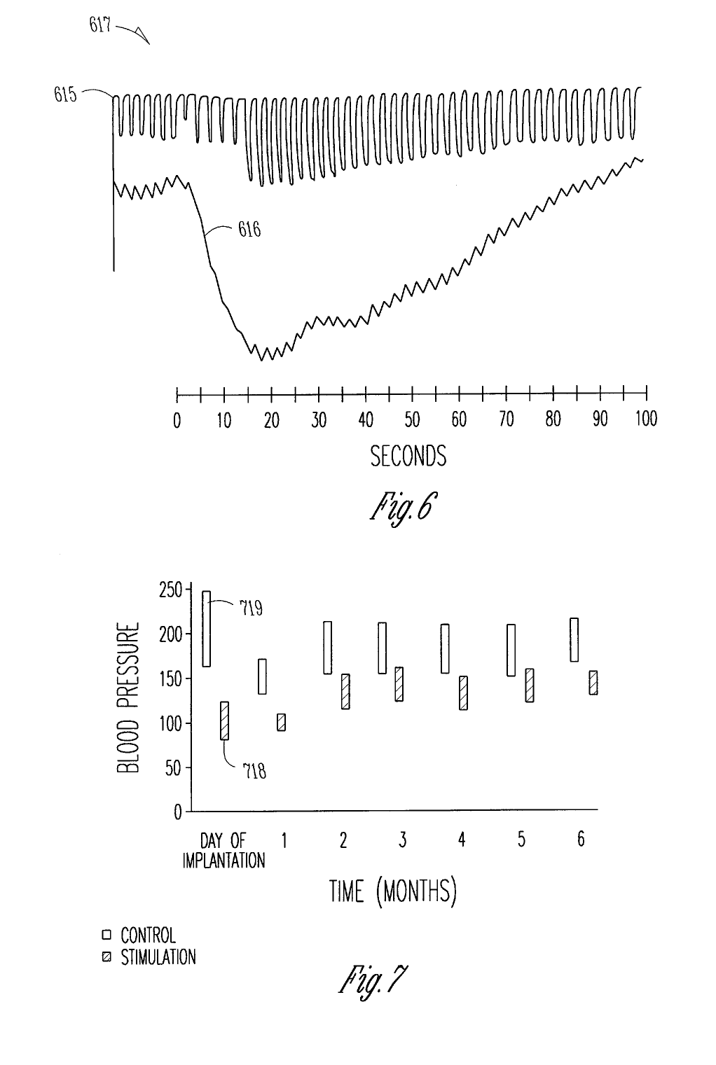

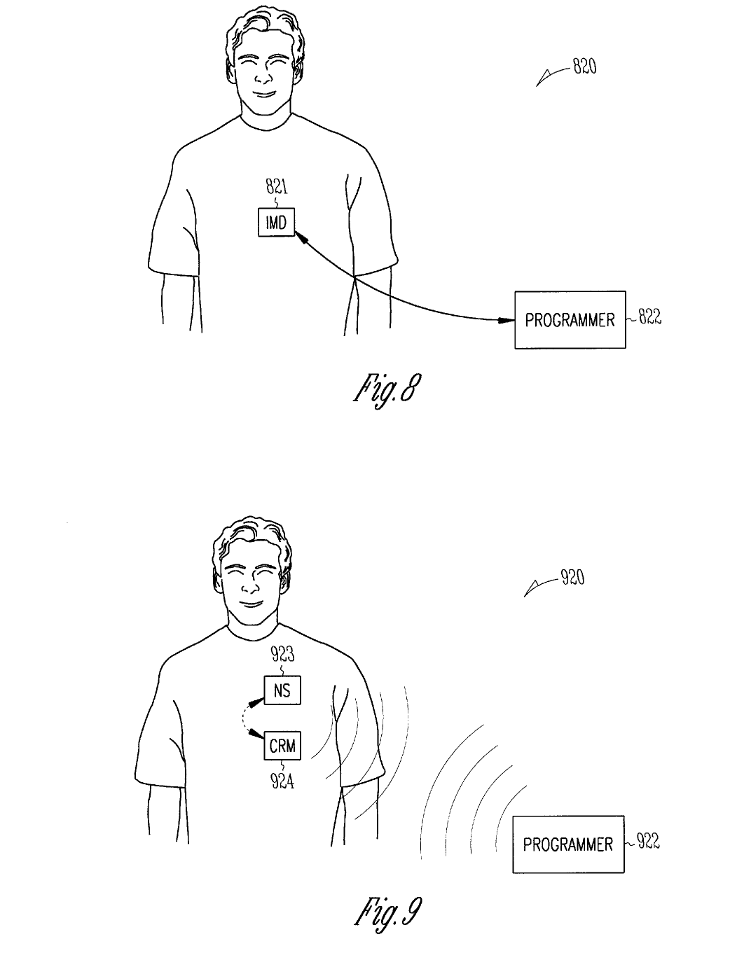

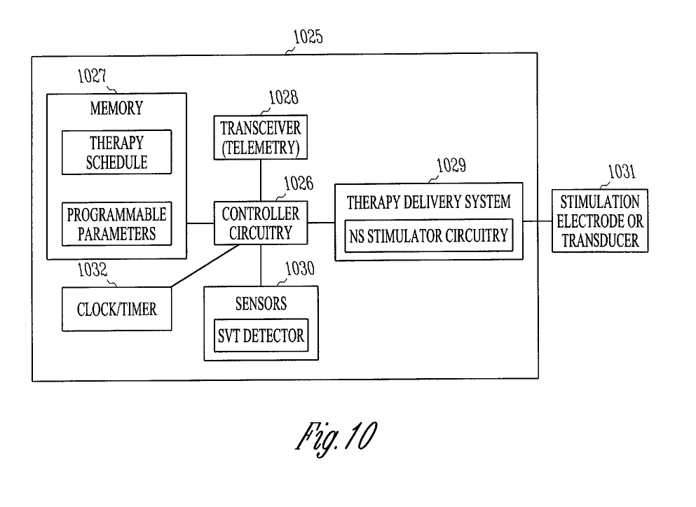

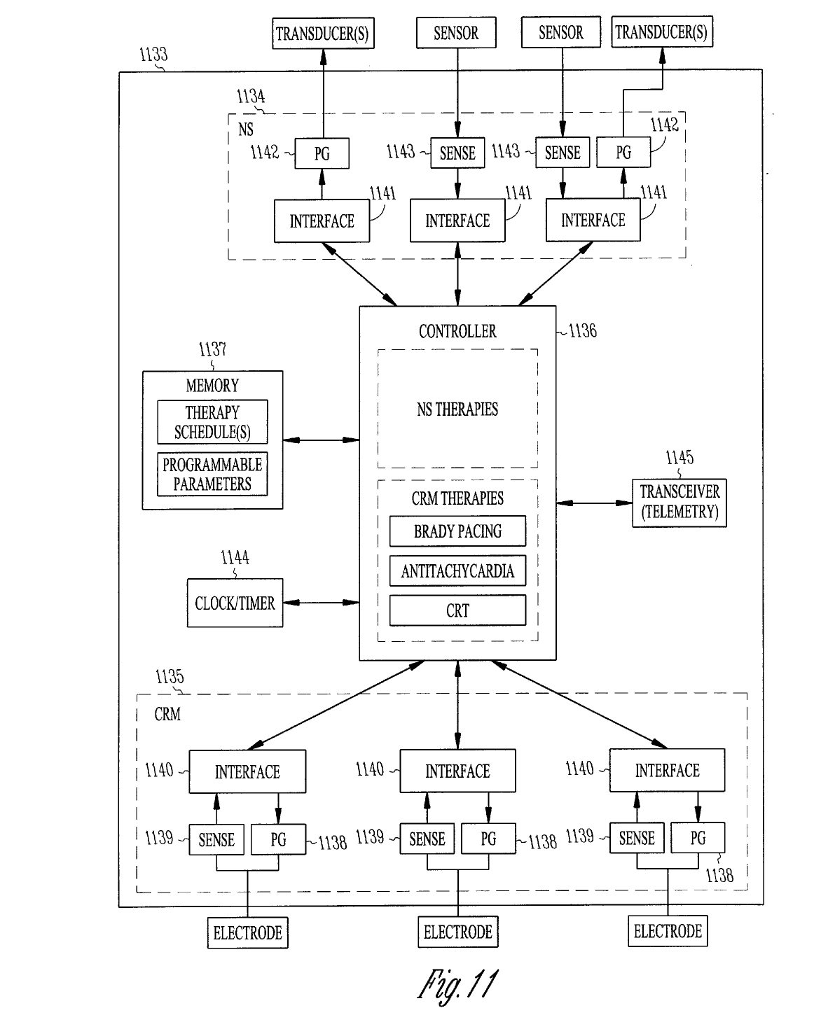

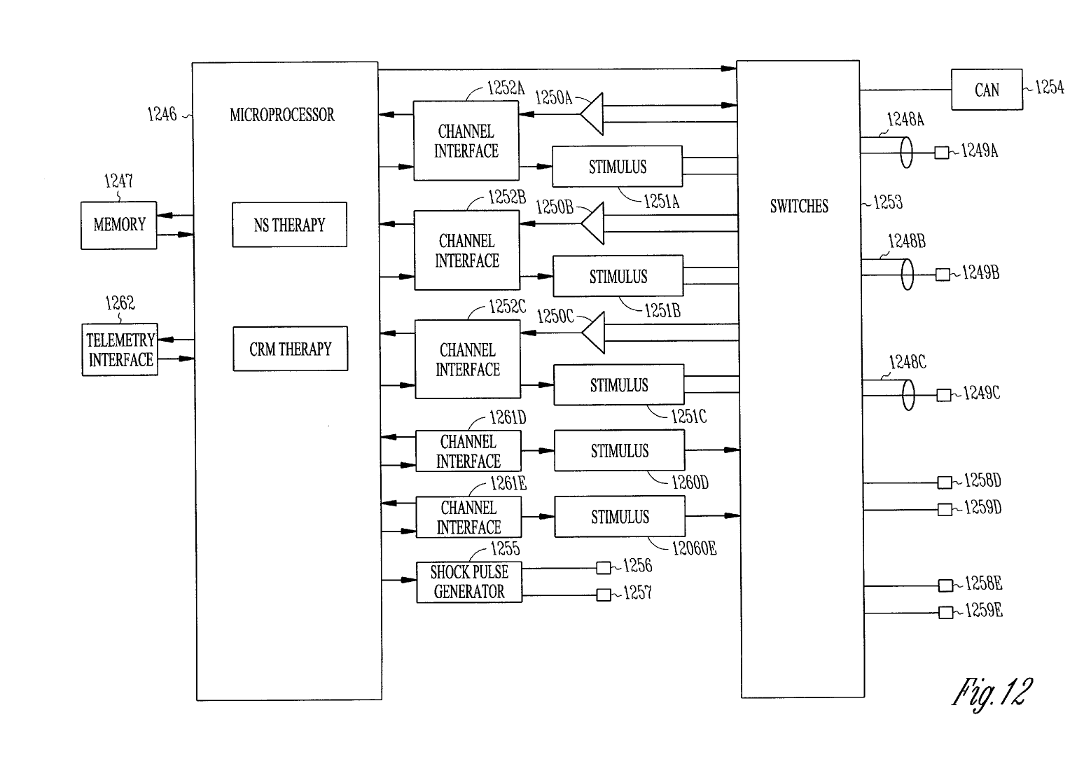

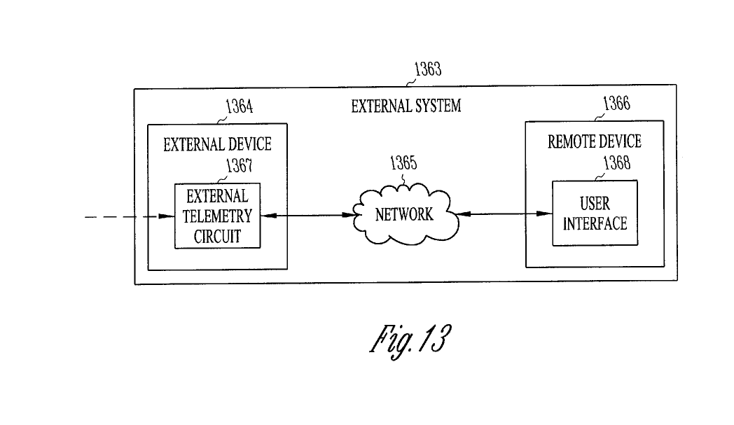

The heart rate and force are increased when the sympathetic nervous system is stimulated, and is decreased when the sympathetic nervous system is inhibited (or the parasympathetic nervous system is stimulated). An afferent nerve conveys impulses toward a nerve center. An efferent nerve conveys impulses away from a nerve center. FIGS. 1A and 1B illustrate neural mechanisms for peripheral vascular control, where FIG. 1A generally illustrates afferent nerves to vasomotor centers and FIG. 1B generally illustrates efferent nerves from vasomotor centers.

Stimulating the sympathetic and parasympathetic nervous systems can have effects other than heart rate and blood pressure. For example, stimulating the sympathetic nervous system dilates the pupil, reduces saliva and mucus production, relaxes the bronchial muscle, reduces the successive waves of involuntary contraction (peristalsis) of the stomach and the motility of the stomach, increases the conversion of glycogen to glucose by the liver, decreases urine secretion by the kidneys, and relaxes the wall and closes the sphincter of the bladder. Stimulating the parasympathetic nervous system (inhibiting the sympathetic nervous system) constricts the pupil, increases saliva and mucus production, contracts the bronchial muscle, increases secretions and motility in the stomach and large intestine, increases digestion in the small intention, increases urine secretion, and contracts the wall and relaxes the sphincter of the bladder. The functions associated with the sympathetic and parasympathetic nervous systems are many and can be complexly integrated with each other.

Vagal modulation may be used to treat a variety of cardiovascular disorders, including but not limited to heart failure, post-MI remodeling, and hypertension. These conditions are briefly described below.

Heart failure refers to a clinical syndrome in which cardiac function causes a below normal cardiac output that can fall below a level adequate to meet the metabolic demand of tissues. Heart failure may present itself as congestive heart failure (CHF) due to the accompanying venous and pulmonary congestion. Heart failure can be due to a variety of etiologies such as ischemic heart disease, hypertension and diabetes.

Hypertension is a cause of heart disease and other related cardiac co-morbidities. Hypertension occurs when blood vessels constrict. As a result, the heart works harder to maintain flow at a higher blood pressure, which can contribute to heart failure. Hypertension generally relates to high blood pressure, such as a transitory or sustained elevation of systemic arterial blood pressure to a level that is likely to induce cardiovascular damage or other adverse consequences. Hypertension has been arbitrarily defined as a systolic blood pressure above 140 mm Hg or a diastolic blood pressure above 90 mm Hg. Consequences of uncontrolled hypertension include, but are not limited to, retinal vascular disease and stroke, left ventricular hypertrophy and failure, myocardial infarction, dissecting aneurysm, and renovascular disease.

Cardiac remodeling refers to a complex remodeling process of the ventricles that involves structural, biochemical, neurohormonal, and electrophysiologic factors, which can result following an MI or other cause of decreased cardiac output. Ventricular remodeling is triggered by a physiological compensatory mechanism that acts to increase cardiac output due to so-called backward failure which increases the diastolic filling pressure of the ventricles and thereby increases the so-called preload (i.e., the degree to which the ventricles are stretched by the volume of blood in the ventricles at the end of diastole). An increase in preload causes an increase in stroke volume during systole, a phenomena known as the Frank-Starling principle. When the ventricles are stretched due to the increased preload over a period of time, however, the ventricles become dilated. The enlargement of the ventricular volume causes increased ventricular wall stress at a given systolic pressure. Along with the increased pressure-volume work done by the ventricle, this acts as a stimulus for hypertrophy of the ventricular myocardium. The disadvantage of dilatation is the extra workload imposed on normal, residual myocardium and the increase in wall tension (Laplace's Law) which represent the stimulus for hypertrophy. If hypertrophy is not adequate to match increased tension, a vicious cycle ensues which causes further and progressive dilatation. As the heart begins to dilate, afferent baroreceptor and cardiopulmonary receptor signals are sent to the vasomotor central nervous system control center, which responds with hormonal secretion and sympathetic discharge. It is the combination of hemodynamics, sympathetic nervous system and hormonal alterations (such as presence or absence of angiotensin converting enzyme (ACE) activity) that ultimately account for the deleterious alterations in cell structure involved in ventricular remodeling. The sustained stresses causing hypertrophy induce apoptosis (i.e., programmed cell death) of cardiac muscle cells and eventual wall thinning which causes further deterioration in cardiac function. Thus, although ventricular dilation and hypertrophy may at first be compensatory and increase cardiac output, the processes ultimately result in both systolic and diastolic dysfunction. It has been shown that the extent of ventricular remodeling is positively correlated with increased mortality in post-MI and heart failure patients.

Baroreflex is a reflex triggered by stimulation of a baroreceptor. A baroreceptor includes any sensor of pressure changes, such as sensory nerve endings in the wall of the auricles of the heart, cardiac fat pads, vena cava, aortic arch and carotid sinus, that is sensitive to stretching of the wall resulting from increased pressure from within, and that functions as the receptor of the central reflex mechanism that tends to reduce that pressure. Additionally, a baroreceptor includes afferent nerve trunks, such as the vagus, aortic and carotid nerves, leading from the sensory nerve endings. Stimulating baroreceptors inhibits sympathetic nerve activity (stimulates the parasympathetic nervous system) and reduces systemic arterial pressure by decreasing peripheral vascular resistance and cardiac contractility. Baroreceptors are naturally stimulated by internal pressure and the stretching of the arterial wall.

Some aspects of the present subject matter locally stimulate specific nerve endings in arterial walls rather than stimulate afferent nerve trunks in an effort to stimulate a desired response (e.g. reduced hypertension) while reducing the undesired effects of indiscriminate stimulation of the nervous system. For example, some embodiments stimulate baroreceptor sites in the pulmonary artery. Some embodiments of the present subject matter involve stimulating either baroreceptor sites or nerve endings in the aorta, the chambers of the heart, the fat pads of the heart, and some embodiments of the present subject matter involve stimulating an afferent nerve trunk, such as the vagus, carotid and aortic nerves. Some embodiments stimulate afferent nerve trunks using a cuff electrode, and some embodiments stimulate afferent nerve trunks using an intravascular lead positioned in a blood vessel proximate to the nerve, such that the electrical stimulation passes through the vessel wall to stimulate the afferent nerve trunk.

FIGS. 2A-2C illustrate a heart. FIG. 2A illustrates the heart 201, a superior vena cava 202, an aortic arch 203, and a pulmonary artery 204, which is useful to provide a contextual relationship with the illustrations in FIGS. 3-5. As is discussed in more detail below, the pulmonary artery 204 includes baroreceptors. A lead is capable of being intravascularly inserted through a peripheral vein and through the tricuspid valve into the right ventricle of the heart (not expressly shown in the figure) similar to a cardiac pacemaker lead, and continue from the right ventricle through the pulmonary valve into the pulmonary artery. A portion of the pulmonary artery and aorta are proximate to each other. Various embodiments stimulate baroreceptors in the aorta using a lead intravascularly positioned in the pulmonary artery. Thus, according to various aspects of the present subject matter, the baroreflex is stimulated in or around the pulmonary artery by at least one electrode intravascularly inserted into the pulmonary artery. Alternatively, a wireless stimulating device, with or without pressure sensing capability, may be positioned via catheter into the pulmonary artery. Control of stimulation and/or energy for stimulation may be supplied by another implantable or external device via ultrasonic, electromagnetic or a combination thereof. Aspects of the present subject matter provide a relatively noninvasive surgical technique to implant a baroreceptor stimulator intravascularly into the pulmonary artery.

FIGS. 2B-2C illustrate the right side and left side of the heart, respectively, and further illustrate cardiac fat pads which have nerve endings that elicit a baroreflex response when stimulated. FIG. 2B illustrates the right atrium 267, right ventricle 268, sinoatrial node 269, superior vena cava 202, inferior vena cava 270, aorta 271, right pulmonary veins 272, and right pulmonary artery 273. FIG. 2B also illustrates a cardiac fat pad 274 between the superior vena cava and aorta. Nerve endings in the cardiac fat pad 274 are stimulated in some embodiments using an electrode screwed into the fat pad, and are stimulated in some embodiments using an intravenously-fed lead proximately positioned to the fat pad in a vessel such as the right pulmonary artery or superior vena cava, for example. FIG. 2C illustrates the left atrium 275, left ventricle 276, right atrium 267, right ventricle 268, superior vena cava 202, inferior vena cava 270, aorta 271, right pulmonary veins 272, left pulmonary vein 277, right pulmonary artery 273, and coronary sinus 278. FIG. 2C also illustrates a cardiac fat pad 279 located proximate to the right cardiac veins and a cardiac fat pad 280 located proximate to the inferior vena cava and left atrium. Baroreceptor nerve endings in the fat pad 279 are stimulated in some embodiments using an electrode screwed into the fat pad 279, and are stimulated in some embodiments using an intravenously-fed lead proximately positioned to the fat pad in a vessel such as the right pulmonary artery 273 or right pulmonary vein 272, for example. Baroreceptors in the 280 are stimulated in some embodiments using an electrode screwed into the fat pad, and are stimulated in some embodiments using an intravenously-fed lead proximately positioned to the fat pad in a vessel such as the inferior vena cava 270 or coronary sinus or a lead in the left atrium 275, for example.

FIG. 3 illustrates baroreceptors in the area of the carotid sinuses 305, aortic arch 303 and pulmonary artery 304. The aortic arch 303 and pulmonary artery 304 were previously illustrated with respect to the heart in FIG. 2A. As illustrated in FIG. 3, the vagus nerve 306 extends and provides sensory nerve endings 307 to baroreceptors in the aortic arch 303, in the carotid sinus 305 and in the common carotid artery 310. The glossopharyngeal nerve 308 provides nerve endings 309 to baroreceptors in the carotid sinus 305. These baroreceptors are sensitive to stretching of the wall resulting from increased pressure from within. Activation of these nerve endings reduce pressure. Neural targets in the fat pads and the atrial and ventricular chambers of the heart can elicit a baroreflex response. Cuffs have been placed around afferent nerve trunks, such as the vagal nerve, leading from baroreceptors to vasomotor centers to stimulate the baroreflex. According to various embodiments of the present subject matter, afferent nerve trunks can be stimulated using a cuff or intravascularly-fed lead positioned in a blood vessel proximate to the afferent nerves.

FIG. 4 illustrates baroreceptors in and around a pulmonary artery 404. The superior vena cava 402 and the aortic arch 403 are also illustrated. As illustrated, the pulmonary artery 404 includes a number of baroreceptors 411, as generally indicated by the dark area. Furthermore, a cluster of closely spaced baroreceptors is situated near the attachment of the ligamentum arteriosum 412. FIG. 4 also illustrates the right ventricle 413 of the heart, and the pulmonary valve 414 separating the right ventricle 413 from the pulmonary artery 404. According to various embodiments of the present subject matter, a lead is inserted through a peripheral vein and threaded through the tricuspid valve into the right ventricle, and from the right ventricle 413 through the pulmonary valve 414 and into the pulmonary artery 404 to stimulate baroreceptors in and/or around the pulmonary artery. In various embodiments, for example, the lead is positioned to stimulate the cluster of baroreceptors near the ligamentum arteriosum 412.

FIG. 5 illustrates baroreceptor fields 512 in the aortic arch 503, near the ligamentum arteriosum and the trunk of the pulmonary artery 504. Some embodiments position the lead in the pulmonary artery to stimulate baroreceptor sites in the aorta and/or fat pads, such as are illustrated in FIGS. 2B-2C.

FIG. 6 illustrates a known relationship between respiration 615 and blood pressure 616 when the left aortic nerve is stimulated. When the nerve is stimulated at 617, the blood pressure 616 drops, and the respiration 615 becomes faster and deeper, as illustrated by the higher frequency and amplitude of the respiration waveform. The respiration and blood pressure appear to return to the pre-stimulated state in approximately one to two minutes after the stimulation is removed. Various embodiments of the present subject matter use this relationship between respiration and blood pressure by using respiration as a surrogate parameter for blood pressure.

FIG. 7 illustrates a known blood pressure response to carotid nerve stimulation in a hypertensive dog during 6 months of intermittent carotid nerve stimulation. The figure illustrates that the blood pressure of a stimulated dog 718 is significantly less than the blood pressure of a control dog 719 that also has high blood pressure. Thus, intermittent stimulation is capable of triggering the baroreflex to reduce high blood pressure.

### Therapies

The present subject matter relates to systems, devices and methods for providing neural stimulation, such as vagus nerve stimulation. Various embodiments provide a stand-alone device, either externally or internally, to provide neural stimulation therapy. For example, the present subject matter may deliver anti-remodeling therapy through neural stimulation as part of a post-MI or heart failure therapy. Neural stimulation may also be used in a hypertension therapy and conditioning therapy, by way of example and not limitation. The present subject matter may also be implemented in non-cardiac applications, such as in therapies to treat epilepsy, depression, pain, obesity, hypertension, sleep disorders, and neuropsychiatric disorders. Various embodiments provide systems or devices that integrate neural stimulation with one or more other therapies, such as bradycardia pacing, anti-tachycardia therapy, remodeling therapy, and the like.

Neural Stimulation Therapies

Examples of neural stimulation therapies include neural stimulation therapies for respiratory problems such a sleep disordered breathing, for blood pressure control such as to treat hypertension, for cardiac rhythm management, for myocardial infarction and ischemia, for heart failure, for epilepsy, for depression, for pain, for migraines and for eating disorders and obesity. Many proposed neural stimulation therapies include stimulation of the vagus nerve. This listing of other neural stimulation therapies is not intended to be an exhaustive listing. Neural stimulation can be provided using electrical, acoustic, ultrasound, light, and magnetic therapies. Electrical neural stimulation can be delivered using any of a nerve cuff, intravascularly-fed lead, or transcutaneous electrodes.

A therapy embodiment involves preventing and/or treating ventricular remodeling. Activity of the autonomic nervous system is at least partly responsible for the ventricular remodeling which occurs as a consequence of an MI or due to heart failure. It has been demonstrated that remodeling can be affected by pharmacological intervention with the use of, for example, ACE inhibitors and beta-blockers. Pharmacological treatment carries with it the risk of side effects, however, and it is also difficult to modulate the effects of drugs in a precise manner. Embodiments of the present subject matter employ electrostimulatory means to modulate autonomic activity, referred to as anti-remodeling therapy (ART). When delivered in conjunction with ventricular resynchronization pacing, also referred to as remodeling control therapy (RCT), such modulation of autonomic activity may act synergistically to reverse or prevent cardiac remodeling.

One neural stimulation therapy embodiment involves treating hypertension by stimulating the baroreflex for sustained periods of time sufficient to reduce hypertension. The baroreflex is a reflex that can be triggered by stimulation of a baroreceptor or an afferent nerve trunk. Baroreflex neural targets include any sensor of pressure changes (e.g. sensory nerve endings that function as a baroreceptor) that is sensitive to stretching of the wall resulting from increased pressure from within, and that functions as the receptor of the central reflex mechanism that tends to reduce that pressure. Baroreflex neural targets also include neural pathways extending from the baroreceptors. Examples of nerve trunks that can serve as baroreflex neural targets include the vagus, aortic and carotid nerves.

Myocardial Stimulation Therapies

Various neural stimulation therapies can be integrated with various myocardial stimulation therapies. The integration of therapies may have a synergistic effect. Therapies can be synchronized with each other, and sensed data can be shared between the therapies. A myocardial stimulation therapy provides a cardiac therapy using electrical stimulation of the myocardium. Some examples of myocardial stimulation therapies are provided below.

A pacemaker is a device which paces the heart with timed pacing pulses, most commonly for the treatment of bradycardia where the ventricular rate is too slow. If functioning properly, the pacemaker makes up for the heart's inability to pace itself at an appropriate rhythm in order to meet metabolic demand by enforcing a minimum heart rate. Implantable devices have also been developed that affect the manner and degree to which the heart chambers contract during a cardiac cycle in order to promote the efficient pumping of blood. The heart pumps more effectively when the chambers contract in a coordinated manner, a result normally provided by the specialized conduction pathways in both the atria and the ventricles that enable the rapid conduction of excitation (i.e., depolarization) throughout the myocardium. These pathways conduct excitatory impulses from the sino-atrial node to the atrial myocardium, to the atrio-ventricular node, and thence to the ventricular myocardium to result in a coordinated contraction of both atria and both ventricles. This both synchronizes the contractions of the muscle fibers of each chamber and synchronizes the contraction of each atrium or ventricle with the contralateral atrium or ventricle. Without the synchronization afforded by the normally functioning specialized conduction pathways, the heart's pumping efficiency is greatly diminished. Pathology of these conduction pathways and other inter-ventricular or intra-ventricular conduction deficits can be a causative factor in heart failure, which refers to a clinical syndrome in which an abnormality of cardiac function causes cardiac output to fall below a level adequate to meet the metabolic demand of peripheral tissues. In order to treat these problems, implantable cardiac devices have been developed that provide appropriately timed electrical stimulation to one or more heart chambers in an attempt to improve the coordination of atrial and/or ventricular contractions, termed cardiac resynchronization therapy (CRT). Ventricular resynchronization is useful in treating heart failure because, although not directly inotropic, resynchronization can result in a more coordinated contraction of the ventricles with improved pumping efficiency and increased cardiac output. Currently, a common form of CRT applies stimulation pulses to both ventricles, either simultaneously or separated by a specified biventricular offset interval, and after a specified atrio-ventricular delay interval with respect to the detection of an intrinsic atrial contraction or delivery of an atrial pace.

CRT can be beneficial in reducing the deleterious ventricular remodeling which can occur in post-MI and heart failure patients. Presumably, this occurs as a result of changes in the distribution of wall stress experienced by the ventricles during the cardiac pumping cycle when CRT is applied. The degree to which a heart muscle fiber is stretched before it contracts is termed the preload, and the maximum tension and velocity of shortening of a muscle fiber increases with increasing preload. When a myocardial region contracts late relative to other regions, the contraction of those opposing regions stretches the later contracting region and increases the preload. The degree of tension or stress on a heart muscle fiber as it contracts is termed the afterload. Because pressure within the ventricles rises rapidly from a diastolic to a systolic value as blood is pumped out into the aorta and pulmonary arteries, the part of the ventricle that first contracts due to an excitatory stimulation pulse does so against a lower afterload than does a part of the ventricle contracting later. Thus a myocardial region which contracts later than other regions is subjected to both an increased preload and afterload. This situation is created frequently by the ventricular conduction delays associated with heart failure and ventricular dysfunction due to an MI. The increased wall stress to the late-activating myocardial regions is most probably the trigger for ventricular remodeling. By pacing one or more sites in a ventricle near the infarcted region in a manner which may cause a more coordinated contraction, CRT provides pre-excitation of myocardial regions which would otherwise be activated later during systole and experience increased wall stress. The pre-excitation of the remodeled region relative to other regions unloads the region from mechanical stress and allows reversal or prevention of remodeling to occur. Cardioversion, an electrical shock delivered to the heart synchronously with the QRS complex, and defibrillation, an electrical shock delivered without synchronization to the QRS complex, can be used to terminate most tachyarrhythmias. The electric shock terminates the tachyarrhythmia by simultaneously depolarizing the myocardium and rendering it refractory. A class of CRM devices known as an implantable cardioverter defibrillator (ICD) provides this kind of therapy by delivering a shock pulse to the heart when the device detects tachyarrhythmias. Another type of electrical therapy for tachycardia is anti-tachycardia pacing (ATP). In ventricular ATP, the ventricles are competitively paced with one or more pacing pulses in an effort to interrupt the reentrant circuit causing the tachycardia. Modern ICDs typically have ATP capability, and deliver ATP therapy or a shock pulse when a tachyarrhythmia is detected.

### Systems

Various embodiments of the present subject matter relate to neural stimulator (NS) devices or components. Examples of neural stimulators include, but are not limited to, anti-hypertension (AHT) devices or AHT components that are used to treat hypertension, and devices or components used to provide neural stimulation for a post-MI therapy. Various embodiments of the present subject matter include stand-alone implantable baroreceptor stimulator systems, include implantable devices that have integrated NS and cardiac rhythm management (CRM) components, and include systems with at least one implantable NS device and an implantable CRM device capable of communicating with each other either wirelessly or through a wire lead connecting the implantable devices. Integrating NS and CRM functions that are either performed in the same or separate devices improves aspects of the NS therapy and cardiac therapy by allowing these therapies to work together intelligently.

FIG. 8 illustrates a system 820 including an implantable medical device (IMD) 821 and a programmer 822, according to various embodiments of the present subject matter. Various embodiments of the IMD 821 include neural stimulator functions only, and various embodiments include a combination of NS and CRM functions. The programmer 822 and the IMD 821 are capable of wirelessly communicating data and instructions. In various embodiments, for example, the programmer 822 and IMD 821 use telemetry coils to wirelessly communicate data and instructions. Thus, the programmer can be used to adjust the programmed therapy provided by the IMD 821, and the IMD can report device data (such as battery and lead resistance) and therapy data (such as sense and stimulation data) to the programmer using radio telemetry, for example. According to various embodiments, the IMD 821 stimulates a neural target to provide NS therapy such as AHT therapy. Various embodiments of the IMD 821 stimulate baroreceptors in the pulmonary artery using a lead fed through the right ventricle similar to a cardiac pacemaker lead, and further fed into the pulmonary artery. According to various embodiments, the IMD 821 includes a sensor to sense ANS activity. Such a sensor can be used to perform feedback in a closed loop control system. For example, various embodiments sense surrogate parameters, such as respiration and blood pressure, indicative of ANS activity. According to various embodiments, the IMD further includes cardiac stimulation capabilities, such as pacing and defibrillating capabilities in addition to the capabilities to stimulate baroreceptors and/or sense ANS activity.

FIG. 9 illustrates a system 920 including a programmer 922, an implantable neural stimulator (NS) device 923 and an implantable cardiac rhythm management (CRM) device 924, according to various embodiments of the present subject matter. Various aspects involve a method for communicating between an NS device, and a CRM device or other cardiac stimulator. In various embodiments, this communication allows one of the devices to deliver more appropriate therapy (i.e. more appropriate NS therapy or CRM therapy) based on data received from the other device. Some embodiments provide on-demand communications. In various embodiments, this communication allows each of the devices to deliver more appropriate therapy (i.e. more appropriate NS therapy and CRM therapy) based on data received from the other device. The illustrated NS device and the CRM device are capable of wirelessly communicating with each other, and the programmer is capable of wirelessly communicating with at least one of the NS and the CRM devices. For example, various embodiments use telemetry coils to wirelessly communicate data and instructions to each other. In other embodiments, communication of data and/or energy is by ultrasonic means.

In some embodiments, the NS device stimulates the baroreflex to provide NS therapy, and senses ANS activity directly or using surrogate parameters, such as respiration and blood pressure, indicative of ANS activity. The CRM device includes cardiac stimulation capabilities, such as pacing and defibrillating capabilities. Rather than providing wireless communication between the NS and CRM devices, various embodiments provide a communication cable or wire, such as an intravenously-fed lead, for use to communicate between the NS device and the CRM device.

Various embodiments relate to a system that seeks to deliver electrically mediated NS therapy, such as AHT therapy, to patients. Various embodiments combine a “stand-alone” pulse generator with a minimally invasive, unipolar lead that directly stimulates baroreceptors in the vicinity of the heart, such as in the pulmonary artery. Various embodiments incorporate a simple implanted system that can sense parameters indicative of blood pressure. This system adjusts the therapeutic output (waveform amplitude, frequency, etc.) so as to maintain a desired quality of life. In various embodiments, an implanted system includes a pulse generating device and lead system, the stimulating electrode of which is positioned near endocardial baroreceptor tissues using transvenous implant technique(s).

According to various embodiments, the lead(s) and the electrode(s) on the leads are physically arranged with respect to the heart in a fashion that enables the electrodes to properly transmit pulses and sense signals from the heart, and with respect to baroreceptors or other neural targets to stimulate the baroreflex. As there may be a number of leads and a number of electrodes per lead, the configuration can be programmed to use a particular electrode or electrodes. According to various embodiments, the baroreflex is stimulated by stimulating afferent nerve trunks.

FIG. 10 illustrates an implantable medical device (IMD) 1025, according to various embodiments of the present subject matter. The illustrated IMD 1025 provides neural stimulation signals for delivery to predetermined neural targets to provide a therapy using an elicited neural stimulation response. The illustrated device includes controller circuitry 1026 and memory 1027. The controller circuitry is capable of being implemented using hardware, software, and combinations of hardware and software. For example, according to various embodiments, the controller circuitry includes a processor to perform instructions embedded in the memory to perform functions associated with the neural stimulation therapy. The illustrated device further includes a transceiver 1028 and associated circuitry for use to communicate with a programmer or another external or internal device. Various embodiments have wireless communication capabilities. For example, some transceiver embodiments use a telemetry coil to wirelessly communicate with a programmer or another external or internal device.

The illustrated device further includes a therapy delivery system 1029, such as neural stimulation circuitry. Other therapy delivery systems, such as drug delivery systems, can be also used with the neural stimulation. The illustrated device also includes sensor circuitry 1030. The sensor circuitry can be used to detect parameter(s) useful to determine a cardiac condition or provide feedback for a therapy. Some embodiments use sensor circuitry adapted to detect nerve traffic. Other physiological parameters, such as heart rate, respiration, and blood pressure can be sensed. According to some embodiments, one or more leads are able to be connected to the sensor circuitry and neural stimulation circuitry. Some embodiments use wireless connections between the sensor(s) and sensor circuitry, and some embodiments use wireless connections between the stimulator circuitry and electrodes. According to various embodiments, the neural stimulation circuitry is used to apply electrical stimulation pulses to desired neural targets, such as through one or more stimulation electrodes 1031 positioned at predetermined location(s). Some embodiments use transducers to provide other types of energy, such as ultrasound, light or magnetic energy. The controller circuitry can control the therapy using a therapy schedule in memory, or can compare a target range (or ranges) of the sensed physiological response(s) stored in the memory to the sensed physiological response(s) to appropriately adjust the intensity of the neural stimulation/inhibition. The target range(s) can be programmable.

According to various embodiments using neural stimulation, the stimulation circuitry is adapted to set or adjust any one or any combination of stimulation features. The intensity of a neural stimulation therapy can be adjusted by adjusting one or more stimulation features. Examples of stimulation features include the amplitude, frequency, polarity and wave morphology of the stimulation signal. Examples of wave morphology include a square wave, triangle wave, sinusoidal wave, and waves with desired harmonic components to mimic white noise such as is indicative of naturally-occurring baroreflex stimulation. Some embodiments of the neural stimulation circuitry are adapted to generate a stimulation signal with a predetermined amplitude, morphology, pulse width and polarity, and are further adapted to respond to a control signal from the controller to modify at least one of the amplitude, wave morphology, pulse width and polarity. Some embodiments of the neural stimulation circuitry are adapted to generate a stimulation signal with a predetermined frequency, and are further adapted to respond to a control signal from the controller to modify the frequency of the stimulation signal.

The controller can be programmed to control the neural stimulation delivered by the stimulation circuitry according to stimulation instructions, such as a stimulation schedule, stored in the memory. Neural stimulation can be delivered in a stimulation burst, which is a train of stimulation pulses at a predetermined frequency. Stimulation bursts can be characterized by burst durations and burst intervals. A burst duration is the length of time that a burst lasts. A burst interval can be identified by the time between the start of successive bursts. A programmed pattern of bursts can include any combination of burst durations and burst intervals. A simple burst pattern with one burst duration and burst interval can continue periodically for a programmed period or can follow a more complicated schedule. The programmed pattern of bursts can be more complicated, composed of multiple burst durations and burst interval sequences. The programmed pattern of bursts can be characterized by a duty cycle, which refers to a repeating cycle of neural stimulation ON for a fixed time and neural stimulation OFF for a fixed time.

According to some embodiments, the controller controls the neural stimulation generated by the stimulation circuitry by initiating each pulse of the stimulation signal. In some embodiments, the controller circuitry initiates a stimulation signal pulse train, where the stimulation signal responds to a command from the controller circuitry by generating a train of pulses at a predetermined frequency and burst duration. The predetermined frequency and burst duration of the pulse train can be programmable. The pattern of pulses in the pulse train can be a simple burst pattern with one burst duration and burst interval or can follow a more complicated burst pattern with multiple burst durations and burst intervals. In some embodiments, the controller 1026 controls the stimulation circuitry 1029 to initiate a neural stimulation session and to terminate the neural stimulation session. The burst duration of the neural stimulation session under the control of the controller 1026 can be programmable. The controller may also terminate a neural stimulation session in response to an interrupt signal, such as may be generated by one or more sensed parameters or any other condition where it is determined to be desirable to stop neural stimulation.

The illustrated device includes a clock or timer 1032 which can be used to execute the programmable stimulation schedule. For example, if a pathological condition and its severity are such that therapy can wait until a more convenient time for the patient, the device can be programmed to enable a therapy for the pathological condition when the pathological condition is detected and to deliver the therapy according to a programmed schedule (e.g. a particular time of day) whenever the therapy is enabled. A stimulation session can begin at a first programmed time, and can end at a second programmed time. Various embodiments initiate and/or terminate a stimulation session based on a signal triggered by a user. Various embodiments use sensed data to enable and/or disable a stimulation session. Thus, for example, the clock can be used to provide an enabling condition for the therapy. By way of another example, two or more conditions may function together to enable a therapy.

According to various embodiments, the schedule refers to the time intervals or period when the neural stimulation therapy is delivered. A schedule can be defined by a start time and an end time, or a start time and a duration.

Various device embodiments apply the therapy according to the programmed schedule contingent on enabling conditions in addition to a detected pathological condition indicated for a neural stimulation therapy, such as patient rest or sleep, low heart rate levels, time of day, and the like. The therapy schedule can also specify how the stimulation is delivered, such as continuously at the pulse frequency throughout the identified therapy period (e.g. 5 Hz pulse frequency for two minutes), or according to a defined duty cycle during the therapy delivery period (e.g. 10 seconds per minute at 5 Hz pulse frequency for two minutes). As illustrated by these examples, the therapy schedule is distinguishable from the duty cycle.

FIG. 11 illustrates an implantable medical device (IMD) 1133 having a neural stimulation (NS) component 1134 and a cardiac rhythm management (CRM) component 1135 according to various embodiments of the present subject matter. The illustrated device includes a controller 1136 and memory 1137. According to various embodiments, the controller includes hardware, software, or a combination of hardware and software to perform the neural stimulation and CRM functions. For example, the programmed therapy applications discussed in this disclosure are capable of being stored as computer-readable instructions embodied in memory and executed by a processor. For example, therapy schedule(s) and programmable parameters can be stored in memory. According to various embodiments, the controller includes a processor to execute instructions embedded in memory to perform the neural stimulation and CRM functions. The illustrated neural stimulation therapy may include predetermined neural stimulation therapies determined to be appropriate for specific pathological conditions, and various combinations of the pathological conditions. For example, the predetermined neural stimulation therapies can include an appropriate therapy for hypertension, an appropriate therapy for ischemia, and an appropriate therapy for a combination of hypertension and ischemia. Various embodiments include CRM therapies, such as bradycardia pacing, anti-tachycardia therapies such as ATP, defibrillation and cardioversion, and cardiac resynchronization therapy (CRT).

The CRM therapy section 1135 includes components, under the control of the controller, to stimulate a heart and/or sense cardiac signals using one or more electrodes. The illustrated CRM therapy section includes a pulse generator 1138 for use to provide an electrical signal through an electrode to stimulate a heart, and further includes sense circuitry 1139 to detect and process sensed cardiac signals. An interface 1140 is generally illustrated for use to communicate between the controller 1136 and the pulse generator 1138 and sense circuitry 1139. Three electrodes are illustrated as an example for use to provide CRM therapy. However, the present subject matter is not limited to a particular number of electrode sites. Each electrode may include its own pulse generator and sense circuitry. However, the present subject matter is not so limited. The pulse generating and sensing functions can be multiplexed to function with multiple electrodes.

The NS therapy section 1134 includes components, under the control of the controller, to stimulate a neural stimulation target and/or sense parameters associated with nerve activity or surrogates of nerve activity such as heart rate, blood pressure and respiration. Three interfaces 1141 are illustrated for use to provide neural stimulation. However, the present subject matter is not limited to a particular number interfaces, or to any particular stimulating or sensing functions. Pulse generators 1142 are used to provide electrical pulses to transducer or transducers for use to stimulate a neural stimulation target. According to various embodiments, the pulse generator includes circuitry to set, and in some embodiments change, the amplitude of the stimulation pulse, the frequency of the stimulation pulse, the burst frequency of the pulse, and the morphology of the pulse such as a square wave, triangle wave, sinusoidal wave, and waves with desired harmonic components to mimic white noise or other signals. Sense circuits 1143 are used to detect and process signals from a sensor, such as a sensor of nerve activity, heart rate, blood pressure, respiration, and the like. The interfaces 1141 are generally illustrated for use to communicate between the controller 1136 and the pulse generator 1142 and sense circuitry 1143. Each interface, for example, may be used to control a separate lead. Various embodiments of the NS therapy section only includes a pulse generator to stimulate a neural target. The illustrated device further includes a clock/timer 1144, which can be used to deliver the programmed therapy according to a programmed stimulation protocol and/or schedule. The illustrated device further includes a transceiver 1145 and associated circuitry for use to communicate with a programmer or another external or internal device. Various embodiments include a telemetry coil.

FIG. 12 shows a system diagram of an embodiment of a microprocessor-based implantable device, according to various embodiments. The controller of the device is a microprocessor 1246 which communicates with a memory 1247 via a bidirectional data bus. The controller could be implemented by other types of logic circuitry (e.g., discrete components or programmable logic arrays) using a state machine type of design. As used herein, the term “circuitry” should be taken to refer to either discrete logic circuitry or to the programming of a microprocessor. Shown in the figure are three examples of sensing and pacing channels designated “A” through “C” comprising bipolar leads with ring electrodes 1248A-C and tip electrodes 1249A-C, sensing amplifiers 1250A-C, pulse generators 1251 A-C, and channel interfaces 1252A-C. Each channel thus includes a pacing channel made up of the pulse generator connected to the electrode and a sensing channel made up of the sense amplifier connected to the electrode. The channel interfaces 1252A-C communicate bidirectionally with the microprocessor 1246, and each interface may include analog-to-digital converters for digitizing sensing signal inputs from the sensing amplifiers and registers that can be written to by the microprocessor in order to output pacing pulses, change the pacing pulse amplitude, and adjust the gain and threshold values for the sensing amplifiers. The sensing circuitry of the pacemaker detects a chamber sense, either an atrial sense or ventricular sense, when an electrogram signal (i.e., a voltage sensed by an electrode representing cardiac electrical activity) generated by a particular channel exceeds a specified detection threshold. Pacing algorithms used in particular pacing modes employ such senses to trigger or inhibit pacing. The intrinsic atrial and/or ventricular rates can be measured by measuring the time intervals between atrial and ventricular senses, respectively, and used to detect atrial and ventricular tachyarrhythmias.

The electrodes of each bipolar lead are connected via conductors within the lead to a switching network 1253 controlled by the microprocessor. The switching network is used to switch the electrodes to the input of a sense amplifier in order to detect intrinsic cardiac activity and to the output of a pulse generator in order to deliver a pacing pulse. The switching network also enables the device to sense or pace either in a bipolar mode using both the ring and tip electrodes of a lead or in a unipolar mode using only one of the electrodes of the lead with the device housing (can) 1254 or an electrode on another lead serving as a ground electrode. A shock pulse generator 1255 is also interfaced to the controller for delivering a defibrillation shock via a pair of shock electrodes 1256 and 1257 upon detection of a shockable tachyarrhythmia.

Neural stimulation channels, identified as channels D and E, are incorporated into the device for delivering stimulation and/or inhibition of neural targets, where one channel includes a bipolar lead with a first electrode 1258D and a second electrode 1259D, a pulse generator 1260D, and a channel interface 1261D, and the other channel includes a bipolar lead with a first electrode 1258E and a second electrode 1259E, a pulse generator 1260E, and a channel interface 1261E. Other embodiments may use unipolar leads in which case the neural stimulation pulses are referenced to the can or another electrode. The pulse generator for each channel outputs a train of neural stimulation pulses which may be varied by the controller as to amplitude, frequency, duty-cycle, and the like. In this embodiment, each of the neural stimulation channels uses a lead which can be intravascularly disposed near an appropriate neural target. Other types of leads and/or electrodes may also be employed. A nerve cuff electrode may be used in place of an intravascularly disposed electrode to provide neural stimulation. In some embodiments, the leads of the neural stimulation electrodes are replaced by wireless links.

The figure illustrates a telemetry interface 1262 connected to the microprocessor, which can be used to communicate with an external device. The illustrated microprocessor 1246 is capable of performing neural stimulation therapy routines and myocardial (CRM) stimulation routines. Examples of NS therapy routines include hypertension, ischemia, post-MI, and heart failure remodeling therapies. Examples of myocardial therapy routines include bradycardia pacing therapies, anti-tachycardia shock therapies such as cardioversion or defibrillation therapies, anti-tachycardia pacing therapies (ATP), and cardiac resynchronization therapies (CRT).

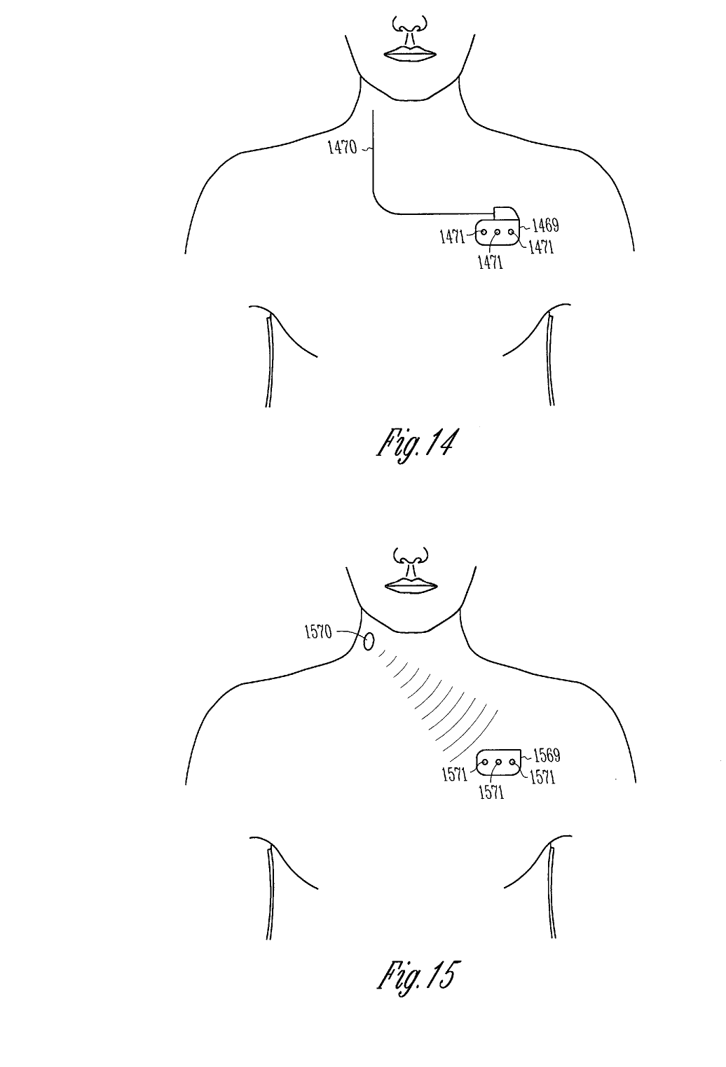

FIG. 13 is a block diagram illustrating an embodiment of an external system 1363. The external system includes a programmer, in some embodiments. In the illustrated embodiment, the external system includes a patient management system. As illustrated, the external system 1363 is a patient management system including an external device 1364, a telecommunication network 1365, and a remote device 1366. External device 1364 is placed within the vicinity of an implantable medical device (IMD) and includes external telemetry system 1367 to communicate with the IMD. Remote device(s) 1366 is in one or more remote locations and communicates with external device 1364 through network 1365, thus allowing a physician or other caregiver to monitor and treat a patient from a distant location and/or allowing access to various treatment resources from the one or more remote locations. The illustrated remote device 1366 includes a user interface 1368. According to various embodiments, the external device includes a programmer or other device such as a computer, a personal data assistant or phone. The external device 1364, in various embodiments, includes two devices adapted to communicate with each other over an appropriate communication channel, such as a computer and a Bluetooth enabled portable device (e.g. personal digital assistant, phone), by way of example and not limitation.

Advanced patient management (APM) systems can be used to enable the patient and/or doctor to adjust parameter(s) to avoid observed or sensed habituation, or to adjust therapy intensity. The inputs can be provided by computers, programmers, cell phones, personal digital assistants, and the like. The patient can call a call center using a regular telephone, a mobile phone, or the internet. The communication can be through a repeater. In response, the call center (e.g. server in call center) can automatically send information to the device to adjust or titrate the therapy. The call center can inform the patient's physician of the event. A device interrogation can be automatically triggered. The results of the device interrogation can be used to determine if and how the therapy should be adjusted and/or titrated to improve the transient response. A server can automatically adjust and/or titrate the therapy using the results of the device interrogation. Medical staff can review the results of the device interrogation, and program the device through the remote server to provide the desired therapy adjustments and/or titrations. The server can communicate results of the device interrogation to the patient's physician, who can provide input or direction for adjusting and/or titrating the therapy.

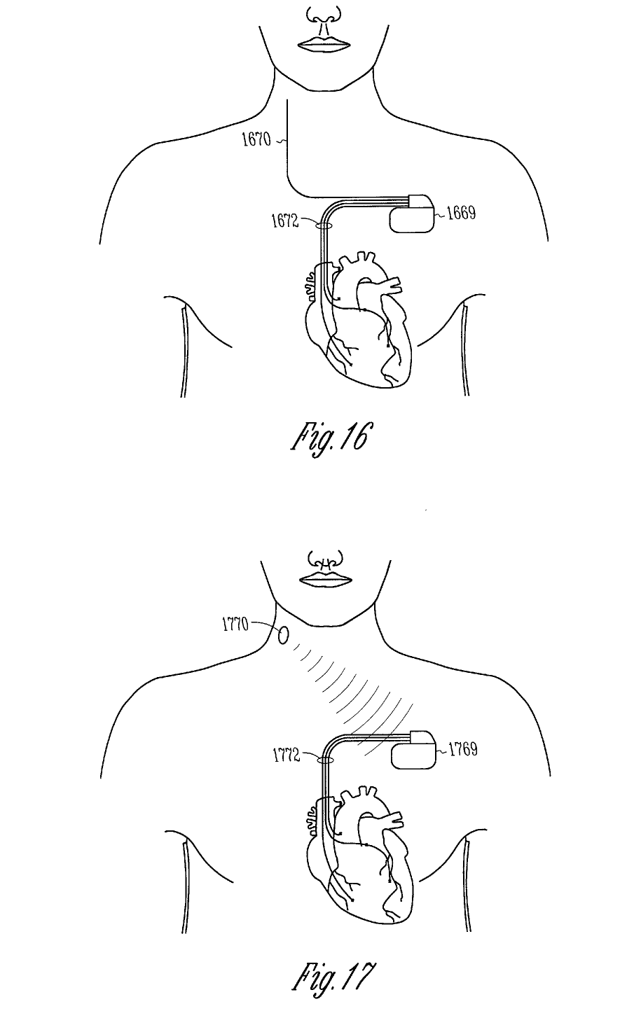

FIG. 14 illustrates a system embodiment in which an implantable medical device (IMD) 1469 is placed subcutaneously or submuscularly in a patient's chest with lead(s) 1470 positioned to stimulate a neural target in the cervical region (e.g. a vagus nerve or cardiac sympathetic nerve). According to various embodiments, neural stimulation lead(s) 1470 are subcutaneously tunneled to a neural target, and can have a nerve cuff electrode to stimulate the neural target. Some vagus nerve stimulation lead embodiments are intravascularly fed into a vessel proximate to the neural target, and use electrode(s) within the vessel to transvascularly stimulate the neural target. For example, some embodiments stimulate the vagus using electrode(s) positioned within the internal jugular vein, and stimulate the stellate ganglion using electrode(s) positioned within the subelavian and/or innominate veins. The neural targets can be stimulated using other energy waveforms, such as ultrasound and light energy waveforms. Other neural targets can be stimulated, such as cardiac nerves and cardiac fat pads. The illustrated system includes wireless ECG electrodes on the housing of the device. These ECG electrodes 1471 are capable of being used to detect heart rate, for example. Various embodiment provide at least three electrodes for use in providing wireless ECG functions. Various embodiments include an electrode on the can, an electrode on a header, and an electrode on a radio frequency (RF) header that forms an orthogonal vector from it to the center of the can with respect to the vector formed from the existing header to the center of the can.

FIG. 15 illustrates a system embodiment that includes an implantable medical device (IMD) 1569 with satellite electrode(s) 1570 positioned to stimulate at least one cervical neural target (e.g. vagus nerve, cardiac sympathetic nerve, and stellate ganglion). The satellite electrode(s) are connected to the IMD, which functions as the planet for the satellites, via a wireless link. Stimulation and communication can be performed through the wireless link. Examples of wireless links include radiofrequency (RF) links and ultrasound links. Examples of satellite electrodes include subcutaneous electrodes, nerve cuff electrodes and intravascular electrodes. Various embodiments include satellite neural stimulation transducers used to generate neural stimulation waveforms such as ultrasound and light waveforms. The illustrated system includes wireless ECG electrodes on the housing of the device. These ECG electrodes 1571 are capable of being used to detect heart rate, for example. Various embodiment provide at least three electrodes for use in providing wireless ECG functions. Various embodiments include an electrode on the can, an electrode on a header, and an electrode on a radio frequency (RF) header that forms an orthogonal vector from it to the center of the can with respect to the vector formed from the existing header to the center of the can.

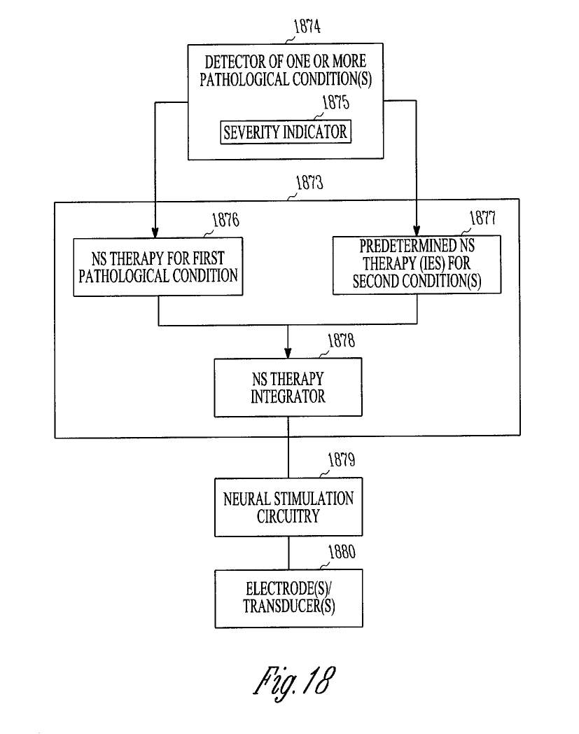

FIG. 16 illustrates an IMD 1669 placed subcutaneously or submuscularly in a patient's chest with lead(s) 1672 positioned to provide a CRM therapy to a heart, and with lead(s) 1670 positioned to stimulate and/or inhibit neural traffic at a cervical neural target, according to various embodiments. According to various embodiments, neural stimulation lead(s) are subcutaneously tunneled to a neural target, and can have a nerve cuff electrode to stimulate the neural target. Some lead embodiments are intravascularly fed into a vessel proximate to the neural target, and use transducer(s) within the vessel to transvascularly stimulate the neural target. For example, some embodiments target the vagus nerve using electrode(s) positioned within the internal jugular vein and some embodiments stimulate the stellate ganglion using electrode(s) positioned within the subclavian and/or innominate veins.

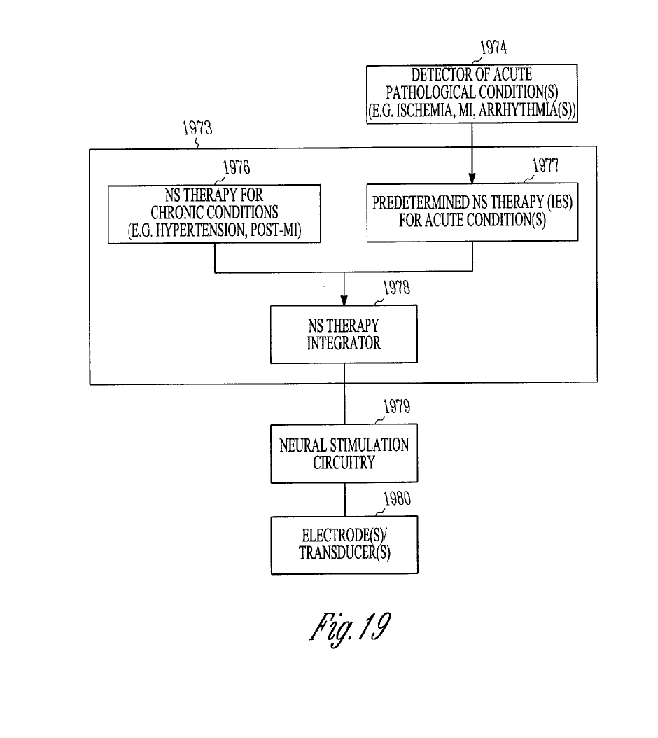

FIG. 17 illustrates an IMD 1769 with lead(s) 1772 positioned to provide a CRM therapy to a heart, and with satellite transducers 1770 positioned to stimulate/inhibit a cervical neural target, according to various embodiments. The satellite transducers are connected to the IMD, which functions as the planet for the satellites, via a wireless link. Stimulation and communication can be performed through the wireless link. Examples of wireless links include RF links and ultrasound links. Although not illustrated, some embodiments perform myocardial stimulation using wireless links. Examples of satellite transducers include subcutaneous electrodes, nerve cuff electrodes and intravascular electrodes.

Those of ordinary skill in the art will understand, upon reading and comprehending this disclosure, that systems can be designed to stimulate only the right vagus nerve, systems can be designed to stimulate only the left vagus nerve, and systems can be designed to bilaterally stimulate both the right and left vagus nerves. Additionally, systems can be designed to stimulate and/or inhibit neural activity of other neural targets. The systems can be designed to stimulate nerve traffic (providing a parasympathetic response when the vagus is stimulated), or to inhibit nerve traffic (providing a sympathetic response when the vagus is inhibited). Various embodiments deliver unidirectional stimulation or selective stimulation of some of the nerve fibers in the nerve.

NS Systems Responsive to Detected Pathological Conditions

Various embodiments automatically adjust neural stimulation (e.g. increase baroreceptor stimulation) upon detection of a pathological condition, such as a pathological cardiac condition. For example, a baroreflex therapy intensity can be increased to increase vasodilatory response and potentially prevent or reduce myocardial ischemic damage. Various embodiments include a feedback mechanism in a cardiac rhythm management device (such as a pacemaker, TCD or CRT device), which also has a stimulation lead for electrically stimulating baroreceptors. The device monitors cardiac electrical activity through existing methods. In the event of an adverse cardiac event such as ventricular fibrillation (VF) and atrial fibrillation (AF), ventricular tachycardia (VT) and atrial tachycardia (AT) above a predefined rate, and dyspnea as detected by a minute ventilation sensor, angina, decompensation and ischemia, the device responds by increasing baroreceptor stimulation up to the maximally allowable level. As a result, blood pressure is temporarily lowered, potentially preventing or reducing myocardial damage due to ischemia. The functionality of a device to treat hypertension, for example, can be expanded if it can respond to adverse cardiac events by temporarily modulating the extent of baroreceptors stimulation. Event detection algorithms for identifying cardiac conditions automatically modulate neural stimulation, allowing an implantable device to respond to the detected cardiac condition by increasing a parasympathetic response, potentially preventing or reducing myocardial ischemic damage.

Following a myocardial infarction, myocytes in the infarcted region die and are replaced by scar tissue, which has different mechanical and elastic properties from functional myocardium. Over time, this infarcted area can thin and expand, causing a redistribution of myocardial stresses over the entire heart. Eventually, this process leads to impaired mechanical function in the highly stressed regions and heart failure. The highly stressed regions are referred to as being heavily “loaded” and a reduction in stress is termed “unloading.”

Various embodiments monitor cardiac electrical activity. Upon detection of a myocardial infarction, the device electrically stimulates the baroreflex, by stimulating baroreceptors in or adjacent to the vessel walls and/or by directly stimulating pressure-sensitive nerves. Increased baroreflex stimulation compensates for reduced baroreflex sensitivity, and improves the clinical outcome in patients following a myocardial infarction. An implantable device (for example, a CRM device) monitors cardiac electrical activity. Upon detection of a myocardial infarction, the device stimulates the baroreflex. Some embodiments of the device stimulate baroreceptors in the pulmonary artery, carotid sinus, or aortic arch with an electrode placed in or adjacent to the vessel wall. In various embodiments, afferent nerves such as the aortic nerve are stimulated directly with a cuff electrode, or with a lead intravenously placed near the afferent nerve. Afferent nerves such as the carotid sinus nerve or vagus nerve are stimulated directly with a cuff electrode, or with a lead intravenously placed near the afferent nerve. In various embodiments, a cardiac fat pad is stimulated using an electrode screwed into the fat pad, or a lead intravenously fed into a vessel or chamber proximate to the fat pad.

Baroreflex stimulation quickly results in vasodilation, and a decrease in systemic blood pressure. This compensates for reduced baroreflex sensitivity and reduces myocardial infarction. According to various embodiments, systemic blood pressure, or a surrogate parameter, are monitored during baroreflex stimulation to insure that an appropriate level of stimulation is delivered. Some aspects and embodiments of the present subject matter provides baroreflex stimulation to prevent ischemic damage following myocardial infarction.

FIG. 18 illustrates a device embodiment configured to integrate neural stimulation therapies for at least two detected pathological conditions. The illustrated device includes a controller 1873 and a detector of one or more pathological conditions 1874. The detector 1874 monitors for the occurrence of certain pathological conditions by sensing physiologic parameter(s) that can provide an indication of the monitored conditions. The illustrated detector, for example includes means for providing an indicator 1875 of the severity of the detected pathological condition. The severity indicator can be based on an assessment of hemodynamic performance or various discrimination algorithms, such as can be used to discriminate among various types of arrhythmias. This indication of severity can be used to prioritize the therapies to address the more severe pathological condition before addressing other pathological conditions; or if both pathological conditions are simultaneously or near simultaneously treated, providing an appropriate therapy weighted to ensure that the more severe condition is effectively treated. The illustrated controller 1873 is configured to control a neural stimulation therapy for a first pathological condition 1876, which may or may not be detected by the detector 1874, to control a predetermined neural stimulation for at least a second condition 1877 which is detected by the detector 1874, and to integrate 1878 the neural stimulation therapies for the first condition and the at least a second condition. The controller 1873 uses the integrated neural stimulation therapies to control the neural stimulation circuitry 1879 to deliver neural stimulation using electrodes or transducers 1880.

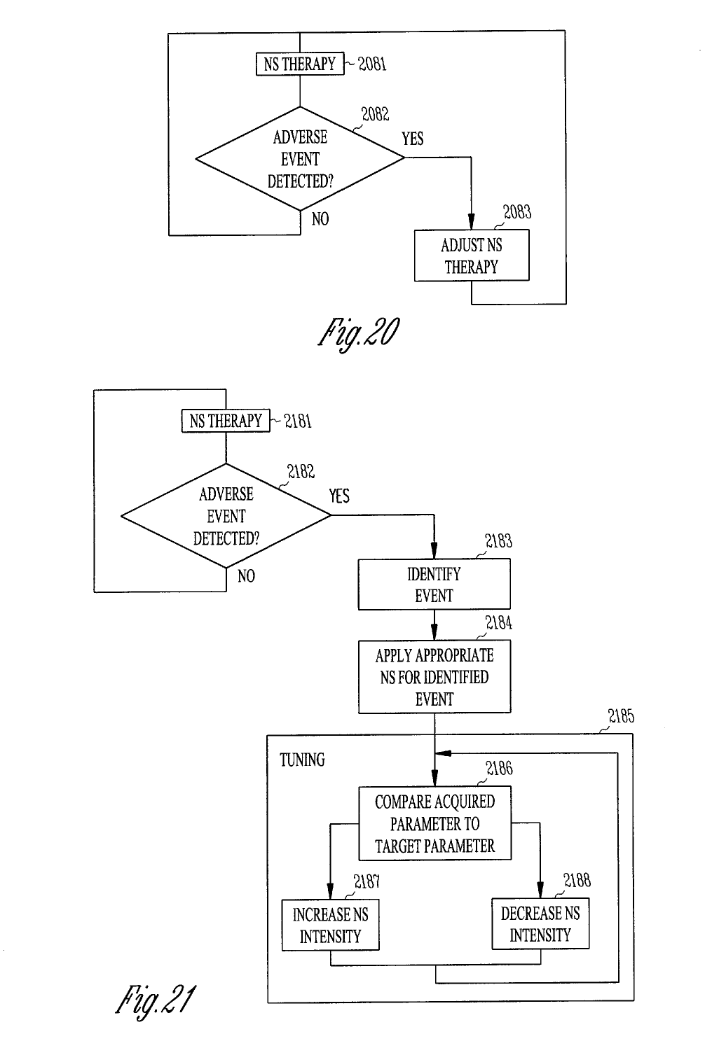

FIG. 19 illustrates a device embodiment configured to integrate a chronic neural stimulation therapy for a chronic pathological condition with a neural stimulation therapy for a detected, acute pathological condition. The illustrated device includes a controller 1973 and a detector of one or more acute pathological conditions 1974, such as ischemia, myocardial infarction, and various arrhythmias. The detector 1974 monitors for the occurrence of certain pathological conditions by sensing physiologic parameter(s) that can provide an indication of the monitored conditions. For example, various detector embodiments are configured to detect ischemia, an acute myocardial infarction, an arrhythmia, a cardiogenic shock, and/or an onset of heart failure decompensation. There are a number of causes for a cardiogenic shock that lead to decreased ventricular filling. For many of these causes, it is believed to be beneficial to withdraw parasympathetic stimulation. The onset of heart failure decompensation is a condition that includes worsened heart failure symptoms, hemodynamic instability, and pulmonary edema. The onset of heart failure decompensation can be sensed using heart sounds, transthoracic impedance, cardiac pressures, and/or vascular pressures. The illustrated controller 1973 is configured to control a neural stimulation therapy for at least one chronic condition 1976 such as an anti-hypertension therapy, a post-MI therapy, or a heart failure remodeling therapy to control a predetermined neural stimulation for at least one acute pathological condition 1977 which is detected by the detector 1974, and to integrate 1978 the neural stimulation therapies for the chronic and acute pathological conditions. The controller 1973 uses the integrated neural stimulation therapies to control the neural stimulation circuitry 1979 to deliver neural stimulation using electrodes or transducers 1980.

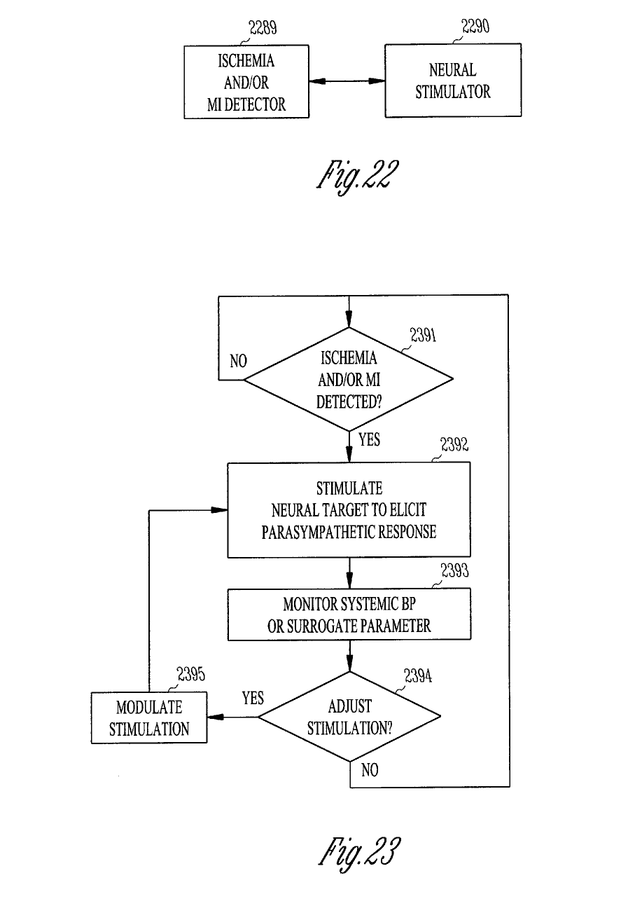

FIGS. 20-21 illustrate methods for modulating baroreceptor stimulation based on detection of a detected cardiac event, according to various embodiments of the present subject matter. The detected cardiac event can be determined by a CRM device, an NS device, or an implantable device with NS/CRM capabilities. FIG. 20 illustrates one embodiment for modulating baroreceptor stimulation based on detection of a detected cardiac condition. A first neural stimulation therapy is delivered at 2081. This first therapy may be a chronic therapy to reduce blood pressure for hypertension, or a remodeling therapy performed after an MI or heart failure. At 2082, it is determined whether a cardiac condition has been detected. If a cardiac condition has not been detected, normal neural stimulation (stimulation according to a normal routine) is performed at 2081. If a cardiac event has been detected, modified neural stimulation is performed at 2083. In various embodiments, the maximum allowable baropacing is performed when a cardiac condition is detected. In one embodiment, the baropacing frequency is increased when a cardiac condition is detected. In an embodiment, the baropacing duty cycle is increased when a cardiac condition is detected. An embodiment increases the baropacing intensity when a cardiac condition is detected. Other procedures can be implemented. For example, various embodiments normally apply neural stimulation and withholds neural therapy when a cardiac condition is detected, and various embodiments normally withhold neural therapy and apply neural stimulation when a cardiac condition is detected. FIG. 21 illustrates an embodiment for modulating neural stimulation based on detection of a cardiac condition. A first neural stimulation therapy is delivered at 2181. This first therapy may be a chronic therapy to reduce blood pressure for hypertension, or a remodeling therapy performed after an MI or heart failure. At 2182, it is determined whether a cardiac condition has been detected. If a cardiac event has not been detected, normal neural stimulation (stimulation according to a normal routine) is performed at 2181. If a cardiac condition has been detected, the condition or event is identified at 2183, and the appropriate neural stimulation for the identified adverse event is applied at 2184. For example, proper blood pressure treatment may be different for ventricular fibrillation than for ischemia. Thus, a detected condition may result in a change in intensity (increase or decrease) of the neural stimulation therapy or in withdrawing the neural stimulation therapy for the chronic condition until the acute condition has been treated using neural stimulation or other therapies. According to various embodiments, the desired neural stimulation is tuned for the identified event at 2185. For example, one embodiment compares an acquired parameter to a target parameter at 2186. The neural stimulation intensity can be increased at 2187 or decreased at 2188 based on the comparison of the acquired parameter to the target parameter.

According to various embodiments, an adverse event includes detectable precursors, such that therapy can be applied to prevent cardiac arrhythmia. In some embodiments, an adverse event includes both cardiac events and non-cardiac events such as a stroke. Furthermore, some embodiments identify both arrhythmic and non-arrhythmic events as adverse events.

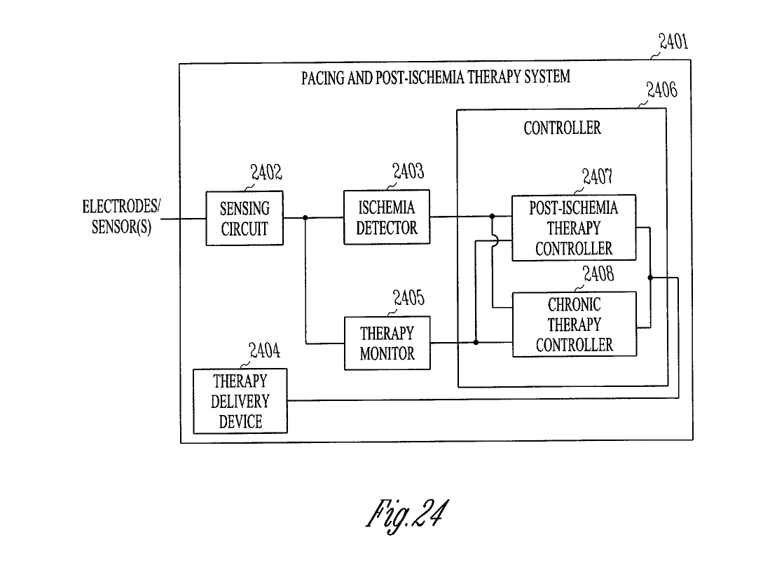

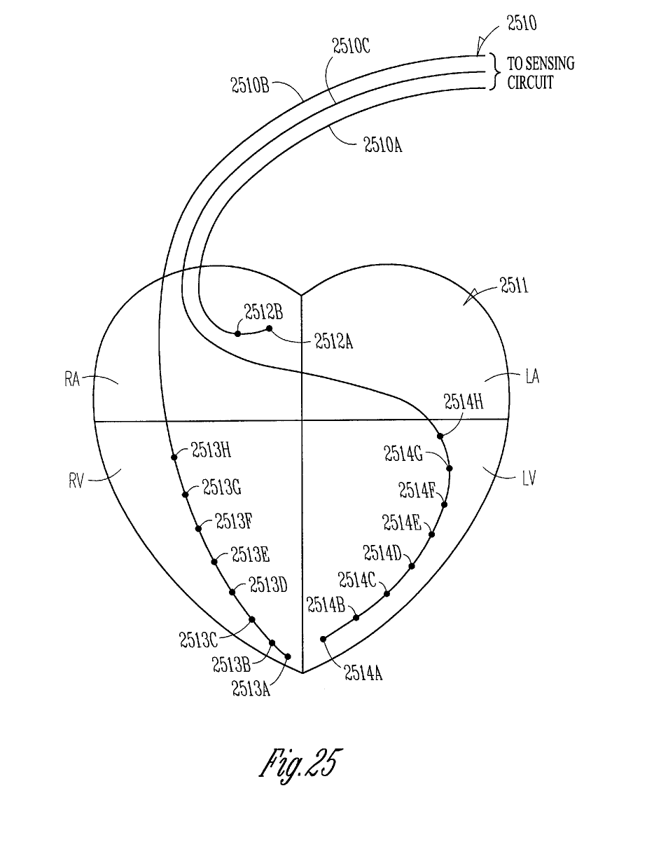

FIGS. 22-23 illustrate a system and method to detect myocardial infarction and perform baropacing in response to the detected myocardial infarction, according to various embodiments of the present subject matter. FIG. 22 illustrates a system that includes a myocardial infarction detector 2289 and a neural stimulator 2290. A myocardial infarction can be detected using an electrocardiogram, for example. For example, a template can be compared to the electrocardiogram to determine a myocardial infarction. Another example detects changes in the ST segment elevation to detect myocardial infarction. In various embodiments, the detector and stimulator are integrated into a single implantable device such as in an AHT device or a CRM device, for example. In various embodiments, the detector and stimulator are implemented in separate implantable devices that are adapted to communicate with each other.

FIG. 23 illustrates a method to detect myocardial infarction and perform neural stimulation in response to the detected myocardial infarction, according to various embodiments of the present subject matter. This method can be performed in response to a myocardial infarction while performing another neural stimulation therapy. At 2391, it is determined whether a myocardial infarction has occurred. Upon determining that a myocardial infarction has occurred, a neural target is stimulated at 2392 to elicit a parasympathetic response. For example, in various embodiments, the baroreceptors in and around the pulmonary artery are stimulated using a lead fed through the right atrium and the pulmonary valve and into the pulmonary artery. Other embodiments stimulate other baroreceptor sites and pressure sensitive nerves. Some embodiments monitor the systemic blood pressure or a surrogate parameter at 2393, and determines at 2394 if the stimulation should be adjusted based on this monitoring. If the stimulation is to be adjusted, the neural stimulation is modulated at 2395. Examples of modulation include changing the amplitude, frequency, burst frequency and/or waveform of the stimulation.

Neural stimulation, such as baroreflex stimulation, can be used to unload after a myocardial infarction. Various embodiments use an acute myocardial infarction detection sensor, such as an ischemia sensor, within a feedback control system of an NS device. In various embodiments, the stimulation lead is implanted through the right atrium and into the pulmonary artery to stimulate baroreceptors in and around the pulmonary artery. Various embodiments implant stimulation cuffs or leads to stimulate afferent nerves, electrode screws or leads to stimulate cardiac fat pads, and leads to stimulate other baroreceptors as provided elsewhere in this disclosure.

Electrical pre-excitation of a heavily loaded region will reduce loading on this region. This pre-excitation may significantly reduce cardiac output resulting in sympathetic activation and an increase in global stress, ultimately leading to deleterious remodeling of the heart. This process may be circumvented by increased neural stimulation to reduce the impact of this reflex. Thus, activation of the parasympathetic nervous system during pre-excitation may prevent the undesirable side-effects of unloading by electrical pre-excitation.

Various embodiments provide an implantable medical device that includes a pre-ischemia neural stimulation therapy and post-ischemia neural stimulation therapy system. Ischemia is used as an example of a detected pathological cardiac condition. Those of ordinary skill in the art will understand, upon reading and understanding this disclosure, how to make appropriate modifications for other detected pathological cardiac conditions such as arrhythmias. For example, an arrhythmia detector can detect predetermined type arrhythmias including bradyarrhythmias and tachyarrhythmias from one or more cardiac signals. In response to the detection of a predetermined type arrhythmia, the controller initiates an anti-arrhythmia therapy. In one embodiment, the controller suspends the neural stimulation therapy, when necessary, to deliver the anti-arrhythmia therapy. For example, in response to a detected tachyarrhythmia, the neural stimulation therapy can be paused to deliver a cardioversion/defibrillation shock pulse and the neural stimulation can be resumed when the tachyarrhythmia is terminated.

In various embodiments, the pre-ischemia and post-ischemia therapy system provides a patient with a long-term neural stimulation therapy and a post-ischemia neural stimulation therapy. The implantable medical device delivers a chronic (long-term) neural stimulation therapy. Examples of such chronic neural stimulation therapy include anti-hypertension therapy and cardiac remodeling control therapy (RCT). The implantable medical device includes a real-time ischemia detector that detects an ischemic state of the patient. The ischemic state indicates occurrences of ischemic event such as acute MI. In response to the occurrence of an ischemic event, the implantable medical device delivers a post-ischemia therapy and, if necessary, adjusts the chronic neural stimulation therapy. The post-ischemia therapy controls or minimizes the damage to the myocardium associated with the ischemic event. A controller provides for adjustment of the chronic neural stimulation therapy and the post-ischemia therapy by feedback control using one or more sensed physiological signals as inputs.

FIG. 24 is a block diagram illustrating an embodiment of a pre-ischemia and post-ischemia therapy system. The illustrated system 2401 includes a sensing circuit 2402, an ischemia detector 2403, a therapy delivery device 2404, a therapy monitor 2405, and a controller 2406.

Sensing circuit senses one or more physiological signals including one or more ischemia-indicating signals and one or more therapy-monitoring signals. In one embodiment, at least one of the one or more physiological signals is both an ischemia-indicating signal and a therapy-monitoring signal. In various embodiments, the one or more therapy-monitoring signals indicate cardiac condition and/or hemodynamic performance, including effects of therapies. Ischemia detector detects the ischemic state from the one or more ischemia-indicating signals sensed by sensing circuit. The ischemic state indicates when an ischemic event is occurring. Therapy delivery device delivers a post-ischemia therapy and/or a pre-ischemia therapy. Therapy monitor produces one or more therapy-monitoring parameters from the one or more therapy-monitoring signals sensed by sensing circuit. The one or more therapy-monitoring parameters include one or more post-ischemia therapy-monitoring parameters and/or one or more pre-ischemia therapy-monitoring parameters. The one or more post-ischemia therapy-monitoring parameters each indicate effectiveness of the post-ischemia therapy. The one or more pre-ischemia therapy-monitoring parameters each indicate effectiveness of the pre-ischemia therapy. In one embodiment, at least one of the therapy-monitoring parameters is used as both a post-ischemia therapy-monitoring parameter and a pre-ischemia therapy-monitoring parameter. Controller includes a post-ischemia therapy controller 2407 and a chronic therapy or pre-ischemia therapy controller 2408. Post-ischemia therapy controller initiates the delivery of the post-ischemia therapy and adjusts the delivery of the post-ischemia therapy based on the ischemic state detected by ischemic detector and the one or more post-ischemia therapy-monitoring parameters produced by therapy monitor. Pre-ischemia therapy controller adjusts the delivery of the chronic therapy based on the ischemic state detected by ischemic detector and the one or more pre-ischemia therapy-monitoring parameters produced by therapy monitor. In one embodiment, the post-ischemia therapy and the chronic therapy are adjusted using the same therapy-monitoring parameter(s) produced by therapy monitor. In another embodiment, the post-ischemia therapy and the pre-ischemia therapy are adjusted using substantially different therapy-monitoring parameters produced by therapy monitor.

Sensing circuit senses the one or more physiological signals through one or more of implantable electrodes/sensors such as endocardial electrodes, epicardial electrodes, and subcutaneous electrodes, impedance sensor, pressure sensor, accelerometer, acoustic sensor such as microphone, strain sensor, and other sensors providing for the sensing of the one or more physiological signals. The one or more physiological signals sensed by sensing circuit include the one or more ischemia-indicating signals used by ischemia detector for detecting the ischemia state and the one or more therapy-monitoring signals used by therapy monitor for producing one or more therapy-monitoring parameters. Examples of such physiological signals include cardiac signals such as electrogram and electrocardiogram (ECG), blood pressure signal, impedance signal, accelerometer signal indicative of heart sounds and/or activity level, acoustic signal indicative of heart sounds, and strain signal indicative of cardiac wall motion.

Ischemia detector detects the ischemic state from the one or more ischemia-indicating signals. Ischemia detector includes an ischemia analyzer running an automatic ischemia detection algorithm to detect the ischemic state from the one or more ischemia-indicating signals. In one embodiment, ischemia detector produces an ischemia alert signal when the ischemic state indicates that an ischemic event, such as an acute MI, has occurred. In an embodiment, the ischemia signal is transmitted to external system for producing an alarm signal and/or a warning message for the patient and/or a physician or other caregiver. In another specific embodiment, implantable medical device produces an alarm signal and/or a warning message for the patient, such as by producing an audible tone or message.

In one embodiment, ischemia detector detects the ischemic state from one or more cardiac signals. Sensing circuit includes a cardiac sensing circuit. In a specific example, cardiac signals are sensed using a wearable vest or a pendant including embedded electrodes configured to sense surface biopotential signals indicative of cardiac activities. The sensed surface biopotential signals are transmitted to implantable medical device via telemetry. In another specific embodiment, ischemia detector detects the ischemic state from one or more wireless ECG signals. Sensing circuit includes a wireless ECG sensing circuit. A wireless ECG is a signal approximating the surface ECG and is acquired without using surface (skin contact) electrodes. An example of a circuit for sensing the wireless ECG is discussed in U.S. patent application Ser. No. 10/795,126, entitled “WIRELESS ECG IN IMPLANTABLE DEVICES,” filed on Mar. 5, 2004, assigned to Cardiac Pacemakers, Inc., which is incorporated herein by reference in its entirety. Examples of wireless ECG-based ischemia detection are is discussed in U.S. patent application Ser. No. 10/955,397, entitled “CARDIAC ACTIVATION SEQUENCE MONITORING AND TRACKING,” filed on Mar. 14, 2005, and U.S. patent application Ser. No. 11/079,744, entitled “CARDIAC ACTIVATION SEQUENCE MONITORING FOR ISCHEMIA DETECTION,” filed on Mar. 14, 2005, both assigned to Cardiac Pacemakers, Inc., which are incorporated herein by reference in their entirety. In another embodiment, ischemia detector 2403 detects the ischemic state from one or more electrogram signals. Sensing circuit 2402 includes an electrogram sensing circuit. Examples of an electrogram-based ischemia detector are discussed in U.S. Pat. No. 6,108,577, entitled, “METHOD AND APPARATUS FOR DETECTING CHANGES IN ELECTROCARDIOGRAM SIGNALS,” and U.S. patent application Ser. No. 09/962,852, entitled “EVOKED RESPONSE SENSING FOR ISCHEMIA DETECTION,” filed on Sep. 25, 2001, both assigned to Cardiac Pacemakers, Inc., which are incorporated herein by reference in their entirety.

Examples of wireless ECG-based ischemia detection are discussed in U.S. patent application Ser. No. 10/955,397 and U.S. patent application Ser. No. 11/079,744. In one embodiment, multiple ECG vectors are sensed to allow ischemia locator to locate the ischemic region by performing a vectorcardiographic analysis. In various embodiments in which multiple wireless ECG vectors are needed, multiple pairs of electrodes are selected, simultaneously or one at a time, for a multi-channel (multi-vector) wireless ECG sensing. The selection of electrode pairs for sensing the ECG vectors is determined by the need of ischemia detector in detecting the ischemic state and the need of ischemia detector in locating the ischemic region. In one embodiment, an ECG vector that provides for a reliable sensing for the purpose of detecting the ischemic state is selected. When two or more ECG vectors provide for the reliable sensing, the ECG vector showing the highest signal-to-noise ratio (SNR) for that purpose is selected. In one embodiment, an optimal linear combination of ECG vectors is formed to provide the highest SNR, such as discussed in U.S. patent application Ser. No. 10/741,814, entitled “SEPARATION OF A SUBCUTANEOUS CARDIAC SIGNAL FROM A PLURALITY OF COMPOSITE SIGNALS,” filed on Dec. 19, 2003, assigned to Cardiac Pacemakers, Inc., which is incorporated herein by reference in its entirety.

In another embodiment, ischemia detector detects the ischemic state from one or more impedance signals. Sensing circuit includes an impedance sensing circuit to sense one or more impedance signals each indicative of a cardiac impedance or a transthoracic impedance. Ischemia detector includes an electrical impedance based sensor using a low carrier frequency to detect the ischemic state from an electrical impedance signal. Tissue electrical impedance has been shown to increase significantly during ischemia and decrease significantly after ischemia, as discussed in Dzwonczyk, et al. IEEE Trans. Biomed. Eng., 51(12): 2206-09 (2004). The ischemia detector senses low frequency electrical impedance signal between electrodes interposed in the heart, and detects the ischemia as abrupt changes in impedance (such as abrupt increases in amplitude or phase angle). In one embodiment, ischemia detector detects the ischemic state from local impedance signals that indicate regional mechanical delays due to slowed activation in an ischemic region.

In another embodiment, ischemia detector detects the ischemic state from one or more signals indicative of heart sounds. Sensing circuit includes a heart sound sensing circuit. The heart sound sensing circuit senses the one or more signals indicative of heart sounds using one or more sensors such as accelerometers and/or microphones. Such sensors are included in implantable medical device or incorporated into lead system. Ischemia detector detects the ischemic state by detecting predetermined type heart sounds, predetermined type heart sound components, predetermined type morphological characteristics of heart sounds, or other characteristics of heart sounds indicative of ischemia. Examples of ischemia detection using heart sounds are discussed in United States Published Application 2006/0282000, entitled ISCHEMIA DETECTION USING HEART SOUND SENSOR, and U.S. application Ser. No. 11/625,003, entitled ISCHEMIA DETECTION USING HEART SOUND TIMINGS, both of which are assigned to Cardiac Pacemakers, Inc. and are incorporated herein by reference in their entirety.

In another embodiment, ischemia detector detects the ischemic state from one or more pressure signals. Sensing circuit includes a pressure sensing circuit coupled to one or more pressure sensors. In a specific embodiment, the pressure sensor is an implantable pressure sensor sensing a signal indicative of an intracardiac or intravascular pressure whose characteristics are indicative of ischemia. Examples of ischemia detection using pressure are discussed in U.S. application Ser. No. 11/624,974, entitled ISCHEMIA DETECTION USING PRESSURE SENSOR, which is assigned to Cardiac Pacemakers, Inc., and is incorporated herein by reference in its entirety.

In another embodiment, ischemia detector detects the ischemic state from one or more accelerometer signals each indicative of regional cardiac wall motion. Sensing circuit includes a cardiac motion sensing circuit coupled to one or more accelerometers each incorporated into a portion of a lead positioned on or in the heart. Ischemia detector detects ischemia as an abrupt decrease in the amplitude of local accelerometer signals or an increase in time delay between local accelerometer signals from different cardiac regions.

In another embodiment, ischemia detector detects the ischemic state from a heart rate variability (HRV) signal indicative of HRV. Sensing circuit includes an HRV sensing circuit to sense the HRV and produce the HRV signal, which is representative of an HRV parameter. HRV is the beat-to-beat variance in cardiac cycle length over a period of time. The HRV parameter includes any parameter being a measure of the HRV, including any qualitative expression of the beat-to-beat variance in cardiac cycle length over a period of time. In a specific embodiment, the HRV parameter includes the ratio of Low-Frequency (LF) HRV to High-Frequency (HF) HRV (LF/HF ratio). The LF HRV includes components of the HRV having frequencies between about 0.04 Hz and 0.15 Hz. The HF HRV includes components of the HRV having frequencies between about 0.15 Hz and 0.40 Hz. The ischemia detector detects ischemia when the LF/HF ratio exceeds a predetermined threshold. An example of an LF/HF ratio-based ischemia detector is discussed in U.S. patent application Ser. No. 10/669,168, entitled “METHOD FOR ISCHEMIA DETECTION BY IMPLANTABLE CARDIAC DEVICE,” filed on Sep. 23, 2003, assigned to Cardiac Pacemakers, Inc., which is incorporated herein by reference in its entirety.

In another embodiment, ischemia detector detects the ischemic state from a signal indicative of cardiac wall motion sensed by one or more strain sensors such as strain gauge sensors each incorporated into lead system to sense a signal indicative of bending forces applied onto a lead. Sensing circuit includes a strain signal sensing circuit coupled to the one or more strain sensors. The timing and amplitude of the bending force reflect the cardiac wall motion in the region where each strain sensor is placed, and such regional cardiac wall motion indicates whether the region is ischemic.

In another embodiment, ischemia detector detects the ischemic state from a signal indicative of changes in blood enzyme levels, such as levels of troponins and creatine-kinases (CK, CK-MB) in blood, as a result of myocardial stress or damage associated with ischemia. Sensing circuit includes a blood enzyme level sensing circuit coupled to an implantable chemoreceptor that detects such changes in blood enzyme levels. Ischemia detector detects ischemia as an abrupt change in a blood enzyme level.

In one embodiment, ischemic detector includes an ischemia locator to locate an ischemic region in heart. The ischemic region indicates the location or the approximate location of ischemic tissue, including infarct tissue, i.e., cardiac tissue whose characteristics are substantially affected by an ischemic event, including acute MI. In various embodiments, ischemia locator uses a plurality of electrodes or sensors to locate the ischemic region by analyzing the signals sensed through these electrodes or sensors.

Controller controls the delivery of the one or more therapies based on the ischemic state, the ischemic region, and the one or more therapy-monitoring parameters. Controller includes a post-ischemia therapy controller and a chronic therapy controller. Post-ischemia therapy controller initiates the delivery of the post-ischemia therapy and adjusts the delivery of the post-ischemia therapy based on the detected ischemic state and the one or more post-ischemia therapy-monitoring parameters. Chronic therapy controller adjusts the delivery of the chronic therapy (before the ischemia is detected) based on the detected ischemic state and the one or more chronic therapy-monitoring parameters. In one embodiment, post-ischemia therapy controller stops the delivery of the post-ischemia therapy when, for example, the detected ischemic state indicates that the ischemic event is no longer occurring and/or the one or more post-ischemia therapy-monitoring parameters no longer indicate a need for the post-ischemia therapy. In one embodiment, the post-ischemia therapy and the chronic therapy are substantially different type therapies. In one embodiment, the post-ischemia therapy and the chronic therapy are the same type therapy but use substantially different parameter(s), and post-ischemia therapy controller initiates the delivery of the post-ischemia therapy by adjusting one or more parameters of the chronic therapy.

Neural stimulation controller initiates a post-ischemia neural stimulation therapy and controls the delivery of neural stimulation pulses from neural stimulation circuit. Examples of post-ischemia neural stimulation therapy are discussed in U.S. patent application Ser. No. 10/745,920, entitled “BAROREFLEX STIMULATION TO TREAT ACUTE MYOCARDIAL INFARCTION,” filed Dec. 24, 2003 (279.705us1—20050149126), and U.S. patent application Ser. No. 11/075,838, entitled “IMPLANTABLE VAGAL STIMULATOR FOR TREATING CARDIAC ISCHEMIA,” filed May 9, 2005 (279.779us1—20060206158), both assigned to Cardiac Pacemakers, Inc., which are incorporated herein by reference in their entirety.

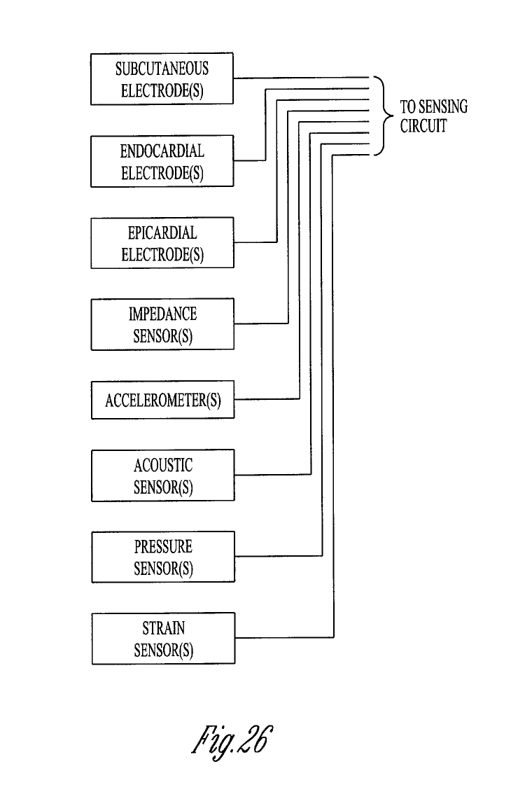

FIG. 25 is an illustration of an embodiment of an electrode system for detecting the ischemic state and/or locating the ischemic region using electrograms and/or impedance signals. The electrode system includes a lead system 2510 that allows for sensing of regional electrograms and/or regional impedances in and/or on heart 2511.

The illustrated lead system includes an atrial lead 2510A, an RV lead 2510B, and a LV lead 2510C. Atrial lead 2510A is an endocardial lead that includes endocardial electrodes 2512A-B for placement in the RA. RV lead 2510B is an endocardial or epicardial lead that includes endocardial or epicardial electrodes 2513A-H for placement in or on the RV. LV lead 2510C is an endocardial or epicardial lead that includes endocardial or epicardial electrodes 2514A-H for placement in or on the LV.

In one embodiment, ischemia detector detects the ischemia state from each of a plurality of electrograms sensed using at least one electrode selected from electrodes 2513A-H and 2514A-H. When the ischemic state indicates the occurrence of an ischemic event, ischemia locator locates the ischemic region by identifying at least one electrode associated with an electrogram from which the occurrence of the ischemic event is detected.

In another embodiment, a plurality of electrodes selected from electrodes 2513A-H and 2514A-H are used to measure impedances. Ischemia detector detects the ischemia state from each measured impedance. When the ischemic state indicates the occurrence of an ischemic event, such as by an abrupt change in the measured impedance, ischemia locator locates the ischemic region by identifying at least one electrode associated with the measured impedance from which the occurrence of the ischemic event is detected.

In a further embodiment, one or more strain sensors are incorporated into each of leads 2510B and 2510C to sense signals indicative of regional cardiac wall motion. Ischemia detector detects the ischemia state from the each of the signals indicative of region cardiac wall motion. When the ischemic state indicates the occurrence of an ischemic event, such as by an abrupt change in the regional cardiac wall motion, ischemia locator locates the ischemic region by identifying at least one strain sensor associated with the signal from which the occurrence of the ischemic event is detected.

In one embodiment, ischemia locator locates the ischemic region by using a combination of methods discussed in this document. In one embodiment, ischemia locator first identifies an approximate ischemic region by analyzing wireless ECG vectors. Then, ischemia locator further locates the ischemic region by analyzing electrograms and impedances sensed from the identified approximate ischemic region. The ischemic region is located by combining the results of localization of all the methods performed, such as by using fuzzy logic.

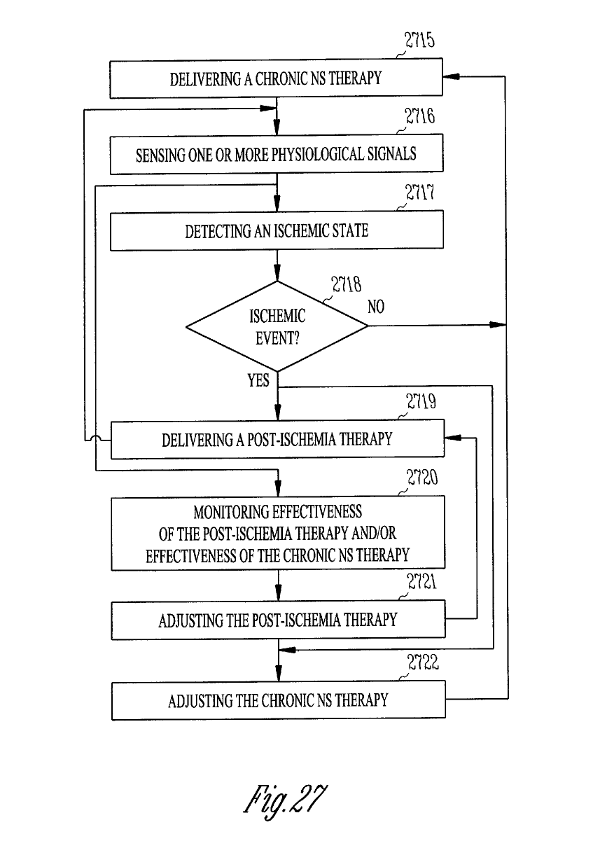

FIG. 26 is an illustration of an embodiment of an electrode/sensor system for detecting the ischemic event and/or locating the ischemic region. In various embodiments, one or more of subcutaneous electrode(s), endocardial electrodes(s), epicardial electrodes, impedance sensor(s), accelerometer(s), acoustic sensor(s), pressure sensor(s), and strain sensor(s) are coupled to sensing circuit to allow sensing of the one or more physiological signals for detecting the ischemic state and monitoring the therapies as discussed in this document. In one embodiment, such electrodes and sensors are each electrically connected to implantable medical device. In another embodiment, one or more of such electrodes and sensors are electrically connected to another device that communicates with implantable medical device via telemetry.

FIG. 27 illustrates a method embodiment for delivering a chronic neural stimulation therapy and a post-ischemia neural stimulation therapy. A chronic neural stimulation therapy is delivered to treat a chronic cardiac condition of a patient at 2715. The patient is diagnosed of a cardiac condition associated with the risk of occurrence of a pathological cardiac condition (e.g. an ischemic event such as an acute MI). The illustrated method uses ischemia as a example of a pathological cardiac condition. Those of ordinary skill in the art will understand, upon reading and comprehending this disclosure, how to provide neural stimulation responsive to other pathological cardiac conditions. In one example, the patient is a heart failure patient. In another example, the patient has suffered an MI and developed heart failure. While delivering a neural stimulation therapy, the patient is monitored for recurring MI.

One or more physiological signals are sensed at 2716. The one or more signals include one or more ischemia-indicating signals that allow for detection of an ischemic state of the patient and one or more therapy-monitoring signals allows for monitoring of therapies delivered to the patient. Examples of the one or more physiological signals include electrogram, wireless ECG signal, blood pressure signal, impedance signal, accelerometer signal indicative of heart sounds and/or activity level, acoustic signal indicative of heart sounds, and strain signal indicative of cardiac wall motion. In one embodiment, at least one physiological signal is used as both an ischemia-indicating signal and a therapy-monitoring signal. In one embodiment, the one or more ischemia-indicating signals and the one or more therapy-monitoring signals include substantially different signals.

The ischemic state is detected at 2717 from the one or more ischemia-indicating signals. The ischemic state indicates the occurrence of each ischemic event. In one embodiment, an ischemic region is located by analyzing the one or more ischemia-indicating signals. The ischemic region includes ischemic or infarct cardiac tissue or is in the proximity of the ischemic or infarct cardiac tissue.

If the ischemia state indicates the occurrence of an ischemic event at 2718, a post-ischemia therapy is delivered at 2719. The effectiveness of the post-ischemia neural stimulation therapy and/or the effectiveness of the chronic neural stimulation therapy are monitored at 2720. One or more therapy-monitoring parameters are produced from the one or more therapy-monitoring signals. Examples of the one or more therapy-monitoring parameters include QRS width, ST-segment deviation, change in dominant orientation vector from wireless ECG, HRV parameter, blood pressure, parameters derived from blood pressure (e.g., rate of pressure change and pulse pressure), regional impedance, amplitude of predetermined type heart sounds (e.g., S3 and S4), magnitude of regional cardiac wall motion, and any other parameters derived from signals sensed by sensing circuit. In one embodiment, at least one post-ischemia therapy-monitoring parameter is produced from a post-ischemia therapy-monitoring signal, and at least one chronic therapy-monitoring parameter is produced from a chronic therapy-monitoring signal. The post-ischemia therapy-monitoring parameter indicates the effectiveness of the post-ischemia therapy. The chronic therapy-monitoring parameter indicates the effectiveness of the chronic therapy.

The post-ischemia neural stimulation therapy is adjusted according to the ischemic state and the one or more therapy-monitoring parameters at 2721. After being initiated in response to the occurrence of the ischemic event, the post-ischemia therapy is adjusted based on the one or more therapy-monitoring parameters. In one embodiment, the post-ischemia therapy is delivered to the located ischemic region. In one embodiment, the delivery of the post-ischemia therapy is stopped when the ischemic state indicate that the ischemic event is no longer occurring and/or when the post-ischemia therapy-monitoring parameter indicates that the post-ischemia therapy is no longer needed.

The chronic therapy is adjusted according to the ischemic state and the one or more therapy-monitoring parameters at 2722. In one embodiment, the chronic therapy is adjusted, to reduce the overall cardiac workload for example, when the ischemic state indicates the occurrence of the ischemic event. In one embodiment, the delivery of the chronic therapy is further adjusted, to restore its pre-ischemia parameters for example, when the ischemic state indicate that the ischemic event is no longer occurring and/or when the post-ischemia therapy-monitoring parameter indicates that the post-ischemia therapy is no longer needed. In one embodiment, the chronic therapy is adjusted using the chronic therapy-monitoring parameter regardless of whether the post-ischemia therapy is being delivered.

In one system embodiment, by way of example and not limitation, the system is configured to deliver a chronic neural stimulation therapy and to detect at least one pathological cardiac condition selected from the following conditions: an arrhythmia, an acute ischemic event, and an acute MI. In response to an arrhythmia such as bradycardia, the system withholds the chronic neural stimulation therapy until the arrhythmic event ceases. In response to an acute ischemia, the system adjusts the chronic neural stimulation therapy to increase a parasympathetic response to reduce heart rate and reduce the cardiac workload during the ischemia event. In response to an acute MI, the system initiates a post-MI neural stimulation therapy to reduce stress on the heart.

Some embodiments, that may or may not be delivering the chronic neural stimulation therapy, prioritize the neural stimulation therapies by ranking the conditions according to severity. For example, the system may rank an arrhythmia as being more severe than an acute ischemic event. In response to detecting both an arrhythmia and an ischemic event, the system provides a neural stimulation therapy that is weighted toward ensuring that the arrhythmic event is terminated. This may involve withdrawing, withholding or titrating the neural therapy for the detected ischemia or may involve adjusting the timing of the neural stimulation therapies. Further, some embodiments integrate neural stimulation therapies for two or more pathological conditions by changing the neural target for one of the therapies when two or more pathological conditions are being treated.

According to various embodiments, the device, as illustrated and described above, is adapted to deliver neural stimulation as electrical stimulation to desired neural targets, such as through one or more stimulation electrodes positioned at predetermined location(s). Other elements for delivering neural stimulation can be used. For example, some embodiments use transducers to deliver neural stimulation using other types of energy, such as ultrasound, light, magnetic or thermal energy.

One of ordinary skill in the art will understand that, the modules and other circuitry shown and described herein can be implemented using software, hardware, and combinations of software and hardware. As such, the terms module and circuitry, for example, are intended to encompass software implementations, hardware implementations, and software and hardware implementations.

The methods illustrated in this disclosure are not intended to be exclusive of other methods within the scope of the present subject matter. Those of ordinary skill in the art will understand, upon reading and comprehending this disclosure, other methods within the scope of the present subject matter. The above-identified embodiments, and portions of the illustrated embodiments, are not necessarily mutually exclusive. These embodiments, or portions thereof, can be combined. In various embodiments, the methods are implemented using a computer data signal embodied in a carrier wave or propagated signal, that represents a sequence of instructions which, when executed by one or more processors cause the processor(s) to perform the respective method. In various embodiments, the methods are implemented as a set of instructions contained on a computer-accessible medium capable of directing a processor to perform the respective method. In various embodiments, the medium is a magnetic medium, an electronic medium, or an optical medium.

The above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. An implantable medical device, comprising:
a detector configured to detect a pathological condition indicated for an acute neural stimulation therapy;
a neural stimulator capable of delivering a chronic neural stimulation therapy and the acute neural stimulation therapy; and
a controller configured to control the neural stimulator to provide the chronic neural stimulation therapy, receive an indicator from the detector that the pathological condition is detected, and control the neural stimulator to integrate the acute neural stimulation therapy with the chronic neural stimulation therapy in response to the indicator.

2. The device of claim 1, wherein the detector is configured to detect:
ischemia;
an acute myocardial infarction;
an arrhythmia;
a cardiogenic shock; or
an onset of heart failure decompensation.

3. The device of claim 1, wherein the chronic neural stimulation therapy includes a heart failure remodeling therapy.

4. The device of claim 1, wherein the chronic neural stimulation therapy includes a post-myocardial infarction therapy.

5. The device of claim 1, wherein the chronic neural stimulation therapy includes an anti-hypertension therapy.

6. An implantable medical device, comprising:
means for performing a first neural stimulation therapy to treat a first pathological condition;
means for detecting a second pathological condition, wherein the second pathological condition is a cardiac condition indicated for a second neural stimulation therapy; and
means for integrating the first neural stimulation therapy and the second neural stimulation therapy into an integrated neural stimulation therapy for the first and second pathological conditions in response to detecting the second pathological condition.

7. The device of claim 6, wherein the second pathological condition includes an arrhythmia, an acute ischemic event, or an acute myocardial infarction.

8. The device of claim 7, wherein the first pathological condition includes hypertension or ventricular remodeling.

9. The device of claim 6, further comprising means for delivering a cardiac rhythm management therapy.

10. A method, comprising:
performing a first neural stimulation therapy to treat a first pathological condition;
detecting a second pathological condition, wherein the second pathological condition is a cardiac condition indicated for a second neural stimulation therapy; and
in response to detecting the second pathological condition, integrating the first neural stimulation therapy and the second neural stimulation therapy into an integrated neural stimulation therapy for the first and second pathological conditions.

11. The method of claim 10, wherein performing the first neural stimulation therapy includes chronically performing the first neural stimulation therapy.

12. The method of claim 11, wherein:
the first pathological condition is hypertension; and
the first neural stimulation therapy includes an anti-hypertension neural stimulation therapy.

13. The method of claim 11, wherein:
the first pathological condition is ventricular remodeling; and
the first neural stimulation therapy includes an anti-remodeling therapy to abate progression of ventricular remodeling.

14. The method of claim 10, wherein detecting the second pathological condition includes detecting an arrhythmia, detecting an acute ischemic event, or detecting an acute myocardial infarction.

15. The method of claim 10, wherein:
the first neural stimulation therapy is an intermittent neural stimulation therapy having neural stimulation times separated by times without neural stimulation; and
integrating the first neural stimulation therapy and the second neural stimulation therapy into an integrated neural stimulation therapy for the first and second pathological conditions includes timing neural stimulation delivered as part of the second neural stimulation therapy to occur between the neural stimulation times of the intermittent neural stimulation therapy.

16. The method of claim 10, wherein integrating the first neural stimulation therapy and the second neural stimulation therapy into an integrated neural stimulation therapy for the first and second pathological conditions includes withdrawing the first neural stimulation therapy until therapy of the second pathological condition is completed.

17. The method of claim 10, wherein integrating the first neural stimulation therapy and the second neural stimulation therapy into an integrated neural stimulation therapy for the first and second pathological conditions includes increasing or decreasing an intensity of the first neural stimulation therapy until therapy of the second pathological condition is completed.

18. The method of claim 10, wherein detecting a second pathological condition including detecting a severity of the second pathological condition, and integrating includes integrating the first neural stimulation therapy and the second neural stimulation therapy based on the severity of the second pathological condition.

19. A method, comprising:
performing a vagal stimulation therapy to treat a chronic pathological condition;
detecting ischemia; and
adjusting the vagal stimulation therapy for the chronic pathological condition in response to detecting the ischemia.

20. The method of claim 19, wherein the vagal stimulation therapy to treat the chronic pathological condition includes a therapy to treat hypertension or a post-myocardial infarction therapy.

21. The method of claim 19, wherein ischemia is a pathological cardiac condition, the method further comprising detecting at least a second pathological condition, wherein adjusting the vagal stimulation therapy includes adjusting the vagal stimulation therapy for the second pathological cardiac condition.

22. The method of claim 21, wherein detecting at least a second pathological condition includes detecting the second pathological condition and a third pathological conduction, wherein adjusting the vagal stimulation therapy includes adjusting the vagal stimulation therapy for the combination of the ischemia, the second pathological condition, and the third pathological condition.

23. The method of claim 19, further comprising detecting an arrhythmia, wherein adjusting the vagal stimulation therapy includes withdrawing the vagal stimulation therapy in response to detecting the arrhythmia.

24. The method of claim 19, further comprising detecting an acute myocardial infarction, wherein adjusting the vagal stimulation therapy includes responding to the detected myocardial infarction with stimulation of the vagus nerve.
